(19) United States
(12) Patent Application Publication
(10) Pub. No.: US 2011/0270151 A1
(43) Pub. Date: Nov. 3, 2011

(54) IMAGE-GUIDED ENERGY DEPOSITION FOR TARGETED DRUG DELIVERY

(75) Inventor: King Chuen Li, Houston, TX (US)

(73) Assignee: The Methodist Hospital Research Institute, Houston, TX (US)

(21) Appl. No.: 13/062,666

(22) PCT Filed: Sep. 8, 2009
(86) PCT No.: PCT/US09/56264

§ 371 Date: Jun. 8, 2011

Related U.S. Application Data
(60) Provisional application No. 61/095,197, filed on Sep. 8, 2008.

(51) Int. Cl. A61M 5/00; C07H 15/252; A61K 9/127; A61K 31/496; A61K 31/704; A61K 51/04; A61K 51/12; C07D 311/82; A61K 49/00; A61K 9/14; C07K 2/00; C07K 16/00; C12N 9/96; C07H 21/04; C07H 21/02; C07J 41/00; A61P 35/00; A61P 37/02; A61P 25/00; A61P 29/00; A61P 31/00; A61P 3/10; A61P 3/06; C07D 403/06

(52) U.S. Cl. 604/20; 544/370; 536/17.4; 424/450; 514/254.05; 514/34; 424/1.89; 424/1.21; 549/388; 424/9.6; 424/489; 530/402; 530/300; 530/387.1; 435/188; 536/23.1; 536/24.5; 552/502; 604/22

(57) ABSTRACT

Disclosed are compositions and methods for targeted drug delivery using image-guided energy deposition to help localize active compounds to particular sites within the body of an animal. Also provided are compounds and formulations thereof for use in the targeted administration of therapeutically, prophylactically, and/or diagnostically effective amounts of such agents to a population of cells or tissues of a mammal in need thereof.

### BACKGROUND OF THE INVENTION

Field of the Invention

The present invention relates generally to the fields of medicine and pharmaceuticals. More particularly, it concerns compositions and methods for facilitating targeted drug delivery using image-guided energy deposition to localize active compounds at particular sites within the body of an animal. The invention also provides compounds and formulations thereof including imaging agents, diagnostics and therapeutics.

Description of Related Art

It is commonly accepted that once a therapeutic agent, such as a drug or cell, is administered to a subject the biodistribution, therapeutic, and side effects are largely dependent on how the therapeutic agent interacts with the different tissues of the subject's body. Although some work has been done on using chemical or physical techniques to activate drugs in vivo, such as photodynamic therapy and drug activated gene therapy, a systematic development of “remote controlled drugs” or “remote controlled cells” has not been accomplished to date. Similarly, much work has been done in developing the “magic bullet” by using targeting ligands or other technologies to try to direct the therapeutic agents to their intended targets, but far less work has been done to modify the targets so that the “bullets” can hit the target more easily.

The use of hyperthermia, image guided focused ultrasound and drugs for spatial and temporal control of gene expression has been advocated by multiple investigators. Gestwici et al. (2006) have shown that it is possible to synthesize bifunctional molecules with one moiety binding to chaperones and another moiety available to interact with targeted protein, thereby utilizing the bulk of the chaperone protein to block protein-protein interactions. It is desired, however, to better target drug delivery to particular sites within the body of an animal.

### BRIEF SUMMARY OF THE INVENTION

The present invention provides new and useful compositions, as well as methods of employing them that may advantageously increase localization of therapeutic, diagnostic and/or prophylactic agents in selected cells or tissues of an animal in need thereof using stress-inducing methodologies.

In one embodiment of the invention, image-guided energy deposition is utilized in combination with smart design of chemical and biologic agents to achieve increased, or fine, control of therapeutic effects. The invention permits the development of therapeutic agents that can be targeted or localized to selected cells, populations of cells, or tissues within the body of an animal at a selected time, to permit and preferably increase the desired effects on the selected cells or tissues. In particular, the invention facilitates targeted accumulation and/or localized delivery of therapeutic agents at effective localized concentrations that are substantially higher than the full-body maximum tolerated dose (MTD).

Such targeted accumulation of drugs is contemplated to be useful in the administration of a number of therapeutically- and diagnostically-effective compounds, and is particularly contemplated to be useful in the administration of pharmaceutical compounds that have maximum tolerated doses lower than that, which is desired in the selected cells or tissues.

The present compositions and methods find particular utility in the delivery of radionuclides, cytotoxic and chemotherapeutic agents, such as those used in anti-cancer therapies. By providing a localized concentration of the cytotoxic agent at an effective dose substantially higher than the maximum tolerated full-body dose, localized cytotoxicity can be increased, thereby facilitating greater localized pharmacotherapy, while at the same time, keeping the overall systemic dose of the active ingredient within the accepted MTD parameters for administration of such agents.

In certain embodiments, the invention also provides new drug delivery vehicles that may be adapted to provide delivery of a multitude of conventional, as well as yet-to-be discovered active ingredient(s) and pharmaceutically-active molecules.

The present invention also provides methods for producing a localized stress response in a population of cells or in one or more selected tissues within the body of an animal. Such methods generally involve the stimulation, induction, activation, or up-regulation of one or more stress responses in the cells or tissues by application of an external inducing agent.

In one embodiment, the invention provides a bifunctional pharmaceutical composition (and related methods for its use) that includes at least one stress-responsive moiety operably linked to at least one active component having a diagnostic or effect in an animal, wherein the at least one active component includes at least a first diagnostic or therapeutic molecule.

While the stress-responsive moiety may be operably linked to the active component by any conventional method, in certain embodiments, the stress-responsive moiety is linked to the at least one active component by a chemical linker, and preferably, by a chemical linker that is covalently linked to the moiety, to the active component, or to both.

Preferably, the at least one stress-responsive moiety is operably linked to one or more of the diagnostic or therapeutic molecules themselves, and in some embodiments, will be operably linked to at least a population of diagnostic or therapeutic molecules that are included within the active component of the composition. In some embodiments, substantially all of the diagnostic or therapeutic molecules will be operably linked to the stress-responsive moiety.

Likewise, in other embodiments, the at least one stress-responsive moiety is operably linked to a second distinct diagnostic or therapeutic molecule, or, alternatively, is operably linked to a combination thereof.

Preferably, the at least one stress-responsive moiety targets, interacts with, and/or specifically binds to at least a first protein or peptide that is expressed in at least one cell in response to localized stress. Exemplary proteins or peptides include, without limitation, a heat-shock protein, a cold-shock protein, a thermally-activatable protein, an inflammatory response protein, or an oxidative stress protein, or a combination thereof.

The active component of the bifunctional pharmaceutical compositions may include a therapeutic, diagnostic, or prophylactic molecule, or a combination thereof.

Preferably, the at least one stress-responsive moiety binds to a protein, polypeptide, or peptide that is induced, expressed, increased, or upregulated in response to one or more stresses, including, but not limited to, cold, heat, light, oxygen, a redox reagent, a free radical, acoustic energy, radio frequency emission, or laser emission, or a combination thereof.

Preferably, the at least one stress-responsive moiety interacts with, or specifically binds to, a peptide or protein that is responsive to heat-shock stress, inflammation, oxidative stress, or a chemically-mediated stress caused by an alkylating agent, a thiol, menadione, diamide, KO₂, CDNB, or a metal ion component such as copper, or a combination thereof.

For linking the stress-responsive moiety to the active component, exemplary linkers include, without limitation, those selected from the group consisting of:

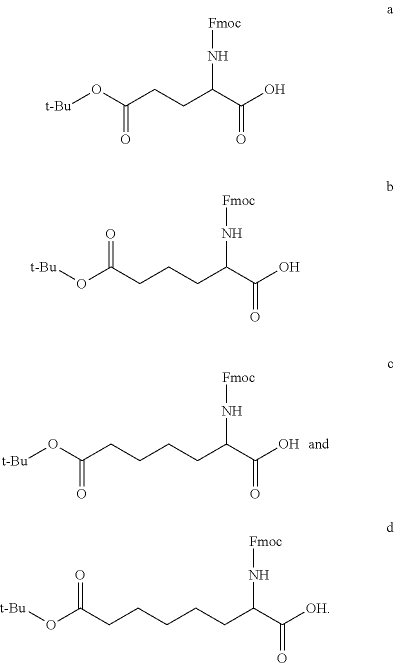

In illustrative embodiments, the first therapeutic molecule is doxorubicin or alternatively, Nutlin-2, a compound which has the formula:

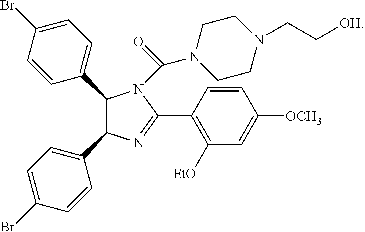

Likewise, in exemplary embodiments, the stress-responsive moiety is a benzoquinone ansamycin, such as geldanamycin (or a derivative or analog thereof); alternatively, a near-infrared fluorescent dye, such as cyanine dye, and particularly, a cyanine 5.5 (Cy5.5) dye (or a derivative or analog thereof); or alternatively still, a compound such as 15-DSG (or a derivative or analog thereof), which has the formula:

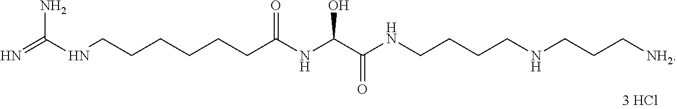

Preferably the bifunctional pharmaceutical compositions of the present invention are adapted and/or configured to release at least a first portion of the at least one active component therefrom by application of an external stress-inducing stimulus such as, without limitation, heat, ultrasound, laser energy, photoacoustic energy, ultrasonography, light energy, radio frequency emission, a magnetic field, or a combination thereof.

The pharmaceutical compositions of the present invention will preferably include at least one active component that includes one or more of an antineoplastic agent, an immunomodulating agent, a neuroactive agent, an antiflammatory agent, an anti-angiogenic agent, a chemotherapeutic, a radiotherapeutic, an antilipidemic agent, a receptor agonist or antagonist, an antiinfective agent, a hormone, a protein, a peptide, an antibody, an antigen binding fragment, an enzyme, an RNA, a DNA, an siRNA, an mRNA, a ribozyme, a cofactor, or a steroid, a detection agent, an imaging agent, a contrast agent, and a gas, or any combination thereof.

The bifunctional pharmaceutical compositions of the invention may optionally further include one or more liposomes, microbubbles, surfactants, lipid complexes, niosomes, ethosomes, transferosomes, phospholipids, sphingosomes, or a combination thereof. 100251 The pharmaceutical compositions disclosed herein may also be contained within one or more nanoparticles, nanoshells, microparticles, nanocapsules, microcapsules, nanospheres, microspheres, or a combination thereof.

The present invention also provides pharmaceutical compositions for use in therapy, and in particular, for use in photoablation, photothermal, photoacoustic, phototherapy, ultrasound, thermal, or laser therapy; as well as compositions for use in diagnosis, and in particular, for use in the diagnosis of a disease, dysfunction, disorder, trauma, injury, or condition, or one or more symptoms thereof.

Exemplary uses for the disclosed compositions in diagnosis include, without limitation, diagnostic imaging modalities such as CT, MRI, PET, ultrasonography and the like.

Use of one or more of the disclosed pharmaceutical compositions in the manufacture of a medicament for diagnosis or therapy is also provided, and in particular, use of such compositions in the manufacture of a medicament for treating a disease, dysfunction, condition, injury, trauma, or disorder, or a symptom thereof, in an animal such as, without limitation, cancer, diabetes, neurological disease, cardiovascular disease, kidney disease, hepatic disease, pulmonary disease, gastrointestinal disease, endocrinological disease or dysfunction, stroke, ischemia, infarction, infection, or sepsis, shock, or any combination thereof.

The invention also provides a method for delivering a therapeutic or diagnostic compound to at least a first cell, population of cells, a tissue, or a collection of tissues in an animal, which comprises providing to an animal in need thereof a therapeutically or diagnostically effective amount of one of the bifunctional pharmaceutical compositions disclosed herein in the presence of a stress-inducing agent for a time sufficient to localize the at least one active component to at least a first cell, population of cells, a tissue, or a collection of tissues within the body of the animal.

The invention also provides a method for providing a diagnostic or imaging component to a selected cell, a population of cells, a first tissue site, or a collection of two or more tissues, within or about the body of an animal, and preferably a mammal, such as a human. This method generally involves at least the step of providing to the animal an effective amount of a bifunctional pharmaceutical composition as disclosed herein, in the presence of a stress-inducing agent under conditions effective to release the diagnostic or imaging component substantially only in the selected cell, population of cells, first tissue site, or collection of two or more tissues within or about the body of the animal.

The compositions of the present invention may be administered systemically, or indirectly, to the target animal, or alternatively, such compositions may be provided locally, or directly, to one or more selected cells, populations of cells, tissues, or collection of tissues, within or about the body of the animal. Preferably, the stress-inducing agent is administered locally to at least a first region of the body that includes the cell, population of cells, first tissue site, or collection of tissues, to which the therapeutic, prophylactic, and/or diagnostic molecule(s) are to be targeted, localized, enriched, or concentrated. In such a method, exemplary stress-inducing agents include, without limitation, the application of one or more of laser energy, photothermal energy, photoacoustic energy, ultrasonography, magnetic resonance energy, radio frequency emission, infrared light, ultraviolet light, visible light, or heat, or any combination thereof to one or more cells, populations of cells, tissues, or collections of tissues within or about the body of the animal undergoing therapy, prophylaxis, and/or diagnosis.

The compositions and methods of the present invention are particularly useful in improving patient outcomes over currently practiced therapies by more effectively providing an effective amount of the selected therapeutic to populations of cells or one or more tissue sites within the body of an animal. In certain circumstances, the present invention may diminish unwanted side effects of conventional therapy. In other embodiments, the administration of a drug targeting compound in accordance with the methods of the invention permit a physician to treat a patient with existing drugs at lower doses (than currently used), preferably while obtaining at least substantially the same or the same efficacy, or alternatively, or to provide an effectively higher localized dose of a therapy beyond the maximum tolerated (systemic) dose of such therapy, and thus ameliorating some or all of the conventional toxic side effects of such drugs. While the exact dosage for a given patient varies from patient to patient, depending on a number of factors including the drug(s) or therapies employed, the particular disease being treated, and the condition and prior history of the patient, such dosages can readily be determined by one or ordinary skill in view of the teachings set forth herein.

### Therapeutic, Prophylactic and Diagnostic Compositions

The inventors contemplate that a wide variety of drug(s), diagnostic reagent(s), and active ingredient(s) that may be employed in the practice of the inventive targeted delivery methods include, but are not limited to, one or more protein(s), peptide(s), polypeptides (including, without limitation, enzymes, antibodies, antigens, antigen binding fragments etc.); RNA molecules (including, without limitation, siRNAs, mRNAs, tRNAs, and catalytic RNAs, such as ribozymes, and the like), DNA molecules (including, without limitation, oligonucleotides, polynucleotides, genes, CDS, introns, exons, plasmids, cosmids, phagemids, baculovirus, vectors [including, without limitation, viral vectors, and such like]); peptide nucleic acids, viral particles, vectors and virions; detection agents, imaging agents, contrast agents, detectable gas, radionuclides, or such like, and pharmaceutically-active molecules, including, without limitation, one or more drugs, pro-drugs, cofactors, ligands, hormones, steroids, targeting domains, linkers, binding domains, catalytic domains, etc., or any combination thereof.

Exemplary active ingredients may include, but are not limited to, one or more antineoplastic agents, cytotoxic agents, transcription factors, apoptotic agents, anti-angiogenics, immunomodulating agents, immunostimulating agents, neuroactive agents, antiflammatory agents, chemotherapeutic agents, antilipidemic agents, hormones, trophic factors, cytokines, receptor agonists or antagonists, antimicrobial agents (including, without limitation, antibacterials, antifungals, antimycotics, antiamebics, antihelminthics, antivirals, and the like), antiinfective agents, or such like, or any combination thereof.

Preferably, drug-delivery formulations disclosed herein will be at least substantially stable at a pH from about 4.2 to about 8.2, and more preferably, will be substantially stable at a pH of from about 5 to about 7.5. Preferably, the active ingredient(s) and targeted drugs will be substantially active at physiological conditions of the animal into which they are being administered.

The compositions of the present invention may also further optionally include one or more liposomes, microbubbles, lipid particles, lipid complexes, or a lipid compound including, but not limited to, those selected from the group consisting of cephalin, ceramide, cerebroside, cholesterol, diacylglycerol, diacylphosphatidylglycerol diacylphosphatidylcholine, diacylphosphatidylethanolamine, phosphatidylethanolamine, phosphatidylcholine, phosphatidylethanolamine, sphingolipid, sphingomyelin, tetraether lipid, or any combination thereof, and may further optionally include one or more binding agents, cell surface active agents, surfactants, lipid complexes, niosomes, ethosomes, transferosomes, phospholipids, sphingolipids, sphingosomes, or any combination thereof, and may optionally be provided within a pharmaceutical formulation that includes one or more nanoparticles, microparticles, nanocapsules, microcapsules, nanospheres, microspheres, or any combination thereof.

The composition may also be formulated to include one or more detectable labels or gases, diagnostic markers, imaging or contrast agents, radiolabeled compound, fluorigenic substance, chemiluminescent or bioluminescent molecule, radioisotope, radionuclides, or any other suitable active ingredient(s) or combinations thereof that may be employed in one or more diagnostic methodologies available in the art based upon the guidance herein.

Preferably, the compounds of the present invention will generally be formulated for systemic and/or localized administration to an animal, or to one or more cells or tissues thereof, and in particular, will be formulated for systemic and/or localized administration to a mammal, or to one or more cells or tissues thereof. In certain embodiments, the compounds and methods disclosed herein will find particular use in the systemic and/or localized administration of one or more of the targeted active agents as described herein to one or more cells or tissues of a human being.

The present invention provides compositions and methods for use in therapy, prophylaxis, and/or diagnosis including, but not limited to, one or more energy transfer modalities such as phototherapy including, without limitation, photoablation, light and laser therapy, thermotherapy (including ultrasonography, acoustic or photoacoustic therapy, magnetic, computer-assisted, and/or radiotherapy), or any combination thereof.

The present invention also provides compositions for use in diagnosis, including, without limitation, the diagnosis of disease via one or more diagnostic imaging modalities (including, without limitation, computer-assisted tomographic [CT] imaging, ultrasonography, magnetic resonance imaging [MRI], positron emission tomography (PET), photoacoustic, and the like). The present invention also provides for the use of one or more of the disclosed pharmaceutical compositions in the manufacture of a medicament for diagnosis, prophylaxis or therapy, and particularly for use in the manufacture of a medicament for diagnosing, treating, and/or preventing one or more diseases, dysfunctions, or disorders in a mammal, and in a human in particular.

The present invention also provides for the use of one or more of the disclosed pharmaceutical compositions in the manufacture of a medicament for diagnosis, prophylaxis or therapy of one or more medical conditions, including, without limitation, cancer; diabetes; neurological disorders; cerebrovascular accidents; stroke; ischemia; infarction; aneurysm; musculoskeletal deficiencies; neuromuscular disorders; peptide, polypeptide, or enzyme deficiency; hormone, cofactor, or trophic factor deficiency; cardiovascular and/or cardiocirculatory disease disorder, or dysfunction; organ disease, dysfunction, or failure; genetic disorders; congenital abnormalities, defects, or malformations; trauma; or such like, or any symptom thereof.

The present invention also provides for the use of one or more of the disclosed pharmaceutical compositions in the manufacture of a medicament for the prevention of disease, including, in the preparation of one or more vaccines suitable for prophylactic administration.

The invention also provides methods for providing a therapeutic, prophylactic, or diagnostic compound to a first cell in a mammal, with the method generally including providing to a mammal in need thereof, an effective amount of at least a first active ingredient for a time effective to provide the desired therapy, prophylaxis or diagnosis in the selected mammal.

In certain aspects of the invention, the invention provides pharmaceutical compositions to facilitate the localized delivery of a therapeutically, prophylactically, or diagnostically-effective dose of one or more compounds to a population of host cells or to one or more tissues or tissue sites within the body of a host animal. In other preferred aspects, the population of host cells or one or more tissues is included within the body of a human, or included within at least a first ex vivo tissue, allograft, transplanted organ, or plurality of cells, tissues, or organ that are compatible for implantation into the body of such a human as part of a typical ex vivo therapy protocol or such like.

### Therapeutic, Prophylactic and Diagnostic Methods

Another important aspect of the present invention concerns methods for using the disclosed compositions to deliver one or more therapeutic agents for treating or ameliorating the symptoms of disease, dysfunction, or deficiency in a mammal. Such methods generally involve administering to a mammal (and in particular, to a human in need thereof), one or more of the disclosed compositions, in an amount and for a time sufficient to treat or ameliorate the symptoms of such a disease, dysfunction, or deficiency in the affected mammal. The methods may also encompass prophylactic treatment of animals suspected of having such conditions, or administration of such compositions to those animals at risk for developing such conditions either following diagnosis, or prior to the onset of symptoms.

### Therapeutic and Diagnostic Kits

Kits including one or more of the disclosed pharmaceutical compositions including a first targeting or localizing moiety; and instructions for using the kit in a therapeutic, diagnostic, and/or other clinical embodiment also represent preferred aspects of the present disclosure. Such kits may further include one or more of the disclosed therapeutic or diagnostic reagents, either alone, or in combination with one or more additional therapeutic compounds, pharmaceuticals, and such like.

The kits of the invention may be packaged for commercial distribution, and may further optionally include one or more delivery devices adapted to deliver the composition(s) to an animal (e.g., syringes, injectables, and the like). Such kits may be therapeutic kits for treating, preventing, or ameliorating the symptoms of a disease, deficiency, dysfunction, and/or injury, and may include one or more of the stress-inducible drug targeting compositions of the invention, and instructions for using the kit in a therapeutic, prophylactic and/or diagnostic regimen.

The container for such kits typically includes at least one vial, test tube, flask, bottle, syringe or other container, into which the pharmaceutical composition(s) may be placed, and preferably suitably aliquotted. Where a second pharmaceutical is also provided, the kit may also contain a second distinct container into which this second composition may be placed. Alternatively, the plurality of pharmaceutical compositions disclosed herein may be prepared in a single mixture, such as a suspension or solution, and may be packaged in a single container, such as a vial, flask, syringe, catheter, cannula, bottle, or other suitable single container.

The kits of the present invention may also typically include a retention mechanism adapated to contain or retain the vial(s) or other container(s) in close confinement for commercial sale, such as, e.g., injection or blow-molded plastic containers into which the desired vial(s) or other container(s) may be retained to minimize or prevent breakage, exposure to sunlight, or other undesirable factors, or to permit ready use of the composition(s) included within the kit.

Alternatively, for the preparation of diagnostic kits, and for methods relating to the use of the disclosed compounds, such kits may be prepared that include at least one pharmaceutical formulation as disclosed herein and instructions for using the composition in diagnosis. The container for such kits may typically include at least one vial, test tube, microcentrifuge tube, or other container, into which the diagnostic composition(s) may be placed and suitably aliquotted. Where a radiolabel or fluorigenic label or other such detecting component is included within the kit, the labeling agent may be provided either in the same container as the pharmaceutical composition, or may alternatively be placed in a second distinct container into which this second composition may be placed and suitably aliquotted. Alternatively, the diagnostic compositions of the present invention may be prepared in combination with one or more additional reagents in a single container, and in most cases, the kit will also typically include a retention mechanism adapted to retain or contain the vial(s) or other container(s) in close confinement for commercial sale and/or convenient packaging and delivery to minimize or avoid any undesirable environmental factors.

### Preparation of Medicaments

Another important aspect of the present invention concerns methods for using the disclosed bifunctional molecules (as well as compositions or formulations including them, which may be referred to herein as “bifunctional compositions”) in the preparation of medicaments for preventing, treating or ameliorating the symptoms of various diseases, dysfunctions, or deficiencies in an animal, such as a vertebrate mammal Use of the disclosed bifunctional compositions is also contemplated in therapy and/or prophylaxis of one or more diseases, disorders, dysfunctions, conditions, disabilities, deformities, or deficiencies, and any symptoms thereof.

Such use generally involves administration to an animal in need thereof one or more of the disclosed compositions that include at least a first therapeutic or prophylactic agent, in an amount and for a time sufficient to prevent, treat, lessen, or ameliorate one or more of a disease, disorder, dysfunction, condition, disability, deformity, or deficiency in the affected animal, or one or more symptoms thereof.

Compositions including one or more of the disclosed pharmaceutical formulations also form part of the present invention, and particularly those compositions that further include at least a first pharmaceutically-acceptable excipient for use in the therapy, prophylaxis, or diagnosis of one or more diseases, dysfunctions, disorders, or such like.

Use of the disclosed compositions is also contemplated, particularly in the manufacture of medicaments and methods involving one or more therapeutic (including chemotherapy, phototherapy, laser therapy, etc.) prophylactic (including e.g., vaccines), or diagnostic regimens, (including, without limitation, in diagnostic imaging, such as CT, MRI, PET, ultrasonography, or the like).

Such formulations may optionally further include one or more additional distinct active ingredients, detection reagents, vehicles, additives or adjuvants, radionuclides, gases, or fluorescent labels as may be suitable for administration to an animal. Such routes of administration are known to and may be selected by those of ordinary skill in the art, and include, without limitation, delivery devices including intramuscular, intravenous, intra-arterial, intrathecal, intracavitary, intraventricular, subcutaneous, or direct injection into an organ, tissue site, or population of cells in the recipient animal.

The use of one or more of the disclosed compositions in the manufacture of a medicament for prophylaxis or therapy of one or more medical conditions is also an important aspect of the invention. Formulation of such compositions for use in administration to an animal host cell, and to a mammalian host cell in particular, is also provided by the invention. In particular embodiments, the invention provides for formulation of such compositions for use in administration to a human, or to one or more selected human host cells, tissues, organs in situ, or to an in vitro or ex situ culture thereof

The present invention also provides for the use of one or more of the disclosed compositions in the manufacture of a medicament or a vaccine for the prophylaxis or prevention of one or more diseases or conditions, including the preparation of one or more vaccines suitable for prophylactic administration to prevent or ameliorate one or more disease symptoms.

The invention also provides methods for providing a therapeutic or prophylactic amount of a compound to a population of cells or to one or more tissues within the body of a mammal, with the method generally including providing to a mammal in need thereof an effective amount of an therapeutic or prophylactic bifunctional composition as disclosed herein that includes at least one targeting or localizing moiety that facilitates the localized accumulation of the therapeutic or prophylactic compound in a selected population of cells, or a selected tissue within the body of the mammal, and for a time effective to provide the desired therapy and/or prophylaxis in the selected cells or tissue of the mammal.

In certain aspects, the invention provides pharmaceutical compositions, and formulations thereof, that are suitable for administration to one or more mammalian host cells. In particular embodiments, the mammalian host cells are preferably human host cells. In other preferred aspects, the host cell is included within the body of a human, or included within at least a first ex vivo tissue or plurality of cells that are compatible for implantation into the body of such a human as part of a typical ex vivo therapy protocol or such like.

The pharmaceutical compositions of the present invention may be administered to a selected animal using any of a number of conventional methodologies, including, without limitation, one or more of parenteral, intravenous, intraperitoneal, subcutaneous, transcutaneous, intradermal, subdermal, transdermal, intramuscular, topical, intranasal, or other suitable route, including, but not limited to, administration, by injection, insertion, inhalation, insufflation, or ingestion.

Yet another advantage of the present invention may include active ingredient(s) and pharmaceutical formulations and compositions that include one or more of such active ingredients useful in treating or ameliorating one or more symptom(s) of an infection or a disease in a mammal. Such methods generally involve administration to a mammal, and in particular, to a human, in need thereof, one or more of the disclosed bifunctional pharmaceutical compositions, in an amount and for a time sufficient to treat, ameliorate, or lessen the severity, duration, or extent of, such a disease or infection in such a mammal.

The methods and compositions of the invention may also be used in prevention, prophylaxis, and/or vaccination of an animal that has, is suspected of having, is at risk for developing, or has been diagnosed with one or more infections and/or diseases, either before, during, or after diagnosis or the onset of one or more clinical symptoms of the disease, or one or more symptoms thereof.

As described in more detail hereinbelow, the disclosed pharmaceutical compositions may be formulated for diagnostic, prophylactic, and/or therapeutic uses, including their incorporation into one or more diagnostic, therapeutic, or prophylactic kits packaged for clinical, diagnostic, and/or commercial resale. The bifunctional compositions disclosed herein may further optionally include one or more detection reagents, one or more additional diagnostic reagents, one or more control reagents, one or more targeting reagents, ligands, binding domains, or such like, and/or one or more therapeutic or imaging compounds, including, without limitation, radionuclides, fluorescent moieties, and such like, or any combination thereof. In the case of diagnostic reagents, the compositions may further optionally include one or more detectable labels that may be used in both in vitro and/or in vivo diagnostic, therapeutic, and/or prophylactic modalities.

### BRIEF DESCRIPTION OF THE DRAWINGS

For promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The following drawings form part of the present specification and are included to demonstrate certain aspects of the present invention. The invention may be better understood by reference to the following description taken in conjunction with the accompanying drawings, in which like reference numerals identify like elements, and in which:

FIG. 1 shows the structure of 15-Deoxyspergulin (15-DSG);

FIG. 2A and FIG. 2B show exemplary synthetic schemes for the preparation of 15-DSG in accordance with the present invention;

FIG. 3 shows the HPLC chromatogram of Compounds 14a and 14b;

FIG. 4 shows the HPLC chromatogram of Compound 16;

FIG. 5 shows an exemplary synthetic scheme for the preparation of FAM-DSG in accordance with the present invention;

FIG. 6 shows the HPLC chromatogram of FAM-DSG;

FIG. 7 shows an exemplary synthetic scheme for the preparation of Cy5.5-DSG in accordance with the present invention;

FIG. 8 shows the HPLC chromatogram of Cy5.5-DSG;

FIG. 9 shows an exemplary synthetic scheme for the preparation of Nutlin-2 in accordance with the present invention;

FIG. 10 shows the HPLC chromatogram of Compound VIII;

FIG. 11 shows the structure of Nutlin-2;

FIG. 12 illustrates the structure of exemplary linkers used in the practice of the invention;

FIG. 13 shows an exemplary synthetic scheme for the preparation of bifunctional molecules in accordance with the present invention;

FIG. 14 shows the HPLC chromatogram of compound B4;

FIG. 15 shows an exemplary synthetic scheme for the preparation of fluorinated, bifunctional molecules in accordance with the present invention;

FIG. 16A, FIG. 16B, FIG. 16C, and FIG. 16D show the immunohistochemistry of HSP70 induction by CuSO₄ in A549 cells. FIG. 16A shows bright field phase-contrast; FIG. 16B shows anti-HSP70 staining; FIG. 16C shows DAPI counterstaining; and FIG. 16D shows an overlay of Anti-HSP and DAPI staining images;

FIG. 17A, FIG. 17B, FIG. 17C, FIG. 17D, FIG. 17E, and FIG. 17F show detection of HSP70 by Cy5.5-DSG in A549 cells, FIG. 17A and FIG. 17B show control at 37° C., bright field and Cy5 filter, respectively; FIG. 17C and FIG. 17D show heat shock at 45° C. for 10 min, bright field and Cy5 filter, respectively; FIG. 17E and FIG. 17F show CuSO₄ induction, bright field and Cy5 filter, respectively;

FIG. 18 shows a local heating apparatus (top oblique view);

FIG. 19 shows a local heating apparatus (top view);

FIG. 20A and FIG. 20B show Mouse 1: heat treatment of the right hind limb at 45° C. for 10 min followed by administration of Cy5.5-DSG. Fluorescence imaging was acquired on a Xenogen IVIS-200. Asymmetric intensity with greater activity localized to the right hind limb on delayed (6 hours) imaging. FIG. 20A: 1 hr post-heat shock; FIG. 20B: 6-hours' post-heat shock;

FIG. 21A and FIG. 21B show Mouse 2: heat treatment of the right hind limb at 45° C. for 10 min followed by administration of Cy5.5-DSG. Relatively increased signal is observed in the right hind limb on delayed imaging (6 hours), FIG. 21A: 1 hr post heat shock; FIG. 21B: 6 hours' post heat shock;

FIG. 22A, FIG. 22B, FIG. 22C, FIG. 22D, and FIG. 22E show DSG-Cy5.5 imaging in mice heat treated in the right hind limb. Immunostaining of induced HSP70. Cy5.5 fluorescence imaging of five mice treated at 45° C. for: (FIG. 22A) 6 min., (FIG. 22B) 7 min, (FIG. 22C) 8 min, (FIG. 22D) 9 min; and (FIG. 22E) 10 min. Cy5.5-DSG was administered at 5 hours post heating. Each mouse was imaged at 5 hr (immediately after injection of Cy5.5-DSG); then at 6, 7, and 8 hours' post-heating;

FIG. 23A and FIG. 23B show immunostaining for HSP70 in soft tissue harvested 6 hours' post-heating at 45° C. for 10 min. (FIG. 23A) Unheated contralateral limb; (FIG. 23B) Heated limb;

FIG. 24 shows a synthetic scheme of various bifunctional molecules according to one aspect of the present invention using different molecules as linkers; and

FIG. 25 shows the synthesis scheme of bifunctional molecule geldanamycin:doxorubicin (GM-TAB-tetraEG-DOX), according to one aspect of the present invention.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

Using image-guided focused ultrasound, the inventor has exploited the induction of chaperone proteins (such as heat shock proteins), which is increased by many times as compared to surrounding unsonicated tissue, using bifunctional molecules with one moiety binding to the upregulated protein and the other to abnormal proteins to block protein-protein interactions that can lead to pathology.

For example, inhibition of the p53-MDM2 interaction with synthetic molecules may lead to the nuclear accumulation and the activation of p53 followed by the death of tumor cells from apoptosis (see e.g., Fry and Vassilev, 2005; Fotouhi and Graves, 2005; and Chene, 2004)^{]}. Ultrasound induced gene expression can also be used in combination with cell therapies. Dendritic cells and stem cells have been used for treating various diseases (see e.g., Alysius et al., 2006; McCurry et al., 2006; Stamm et al., 2006; and Chang et al., 2006). Once the cells have been introduced into the tissue, however, it is very difficult to control their behaviors. Using cell tracking approaches such as iron oxide labeling or reporter genes the inventor is now able to track the cells in vivo (see e.g., Bengel et al., 2006; Bulte and Kraitchman, 2004).

Using promoters that can be triggered by image-guided ultrasound coupled to genes with tissue specificity and genes with desired therapeutic effects such as TNF-α for cytotoxic effect the selectivity and effectiveness of cell therapies can be significantly increased. These are only a few possibilities out of many that can be exploited by combining image guided energy deposition with smart design of chemical and biologic agents for spatial, temporal and tissue specific control of therapies.

### Heat Shock Proteins

Oxidizing reagents can damage critical cellular molecules, including nucleic acids, proteins, and lipids. To protect against oxidant damage, cells contain an array of defense mechanisms. One such mechanism involves heat shock (sometimes referred to as “heat stress”) proteins (HSPs). HSPs are induced by a variety of stimuli including elevated temperature, ischemia, hypoxia, pressure overload, and some chemicals. They help to maintain the metabolic and structural integrity of the cell, as a protective response to external stresses.

Heat stress (typically above normal growth temperature) up-regulates the rapid synthesis of a multigene family of proteins, originally called heat shock proteins, which are the result of a response often referred to as the heat shock response. Prior sub-lethal heat stress transiently increases the ability of a cell to withstand an otherwise lethal subsequent heat challenge. This phenomenon, known as thermotolerance, played a key role in launching numerous studies in both in vitro and in vivo experimental models in which a similar association was found between the heat shock response and protection against either simulated hypoxia or ischemia (for a review see e.g., Benjamin and McMillian, 1998).

Indeed, diverse stresses, including heavy metals, amino acid analogues, inflammation, and oxidative/ischemic stress, induce the expression of HSP genes. Consequently, the terms “stress proteins” or “heat shock family of stress proteins” are preferred, although many of these proteins have essential functions during unstressed conditions.

Stress proteins belong to multigene families that range in molecular size from 10 to 150 kDa and are found in all major cellular compartments. The convention is to name stress proteins of various molecular sizes as follows: Hsp27, Hsp70, and Hsp90; whereas heat shock protein genes are designated as follows: hsp27, hsp70, and hsp90. The distinction between constitutively expressed (e.g., Hsp70 and Hsp90β) or cognate members of the HSP family and their inducible isoforms (Hsp70 and Hsp90α, respectively) is arbitrary, since accumulating evidence, in physiologically relevant in vivo systems, now indicates that such relationships depend on cell- and tissue-restricted expression.

HSPs were originally observed to be expressed in increased amounts in mammalian cells that were exposed to sudden elevations of temperature, while the expression of most cellular proteins is significantly reduced. It has since been determined that such proteins are produced in response to various types of stress, including glucose deprivation. As used herein, the term “heat shock protein” encompasses both proteins that are expressly labeled as such, as well as other stress proteins, including homologues of such proteins that are expressed constitutively (i.e., in the absence of stressful conditions). Examples of HSPs include, but are not limited to BiP (also referred to as grp78), hsp70, hsc70, gp96 (grp94), hsp60, hsp40 and hsp90.

HSPs have the ability to bind other proteins in their non-native states, and in particular to bind nascent peptides emerging from ribosomes or extruded into the endoplasmic reticulum (Hendrick and Hartl, 1993). HSPs have also been shown to play an important role in the proper folding and assembly of proteins in the cytosol, endoplasmic reticulum and mitochondria, and for this reason have been termed “molecular chaperones” (see e.g., Frydman et al., 1994).

### Heat Shock Proteins in Cancer

Heat shock proteins (HSPs) were discovered nearly a half-century ago, and found to be inducible by different kinds of stress (Ritossa, 1962). HSPs behave as molecular chaperones for other cellular proteins and are conserved in both prokaryotes and eukaryotes. HSPs have strong cytoprotective effects and can help in maintaining the proper conformation of other proteins after a large variety of stresses. Stress can be any sudden change in environment such as heat shock, oxidative stress or anti-cancer drugs (see e.g., Schmitt et al., 2007; Soti et al., 2005; Calderwood et al., 2005; Garrido et al., 2006; Ciocca and Calderwood, 2005; and Kregel, 2002). Stress increases the amount of damage to proteins by inhibiting their elimination via the proteasome, as well as by damaging the chaperones themselves. Mammalian HSPs are classified according to their molecular size into five families, HSP100, HSP90, HSP70, HSP60 and the small HSPs. HSPs can be constitutively expressed or regulated inductively and are targeted to different subcellular compartments. HSPs are implicated in tumor cell proliferation, differentiation, invasion, metastasis, death, and recognition by the immune system. There is evidence that HSPs are useful biomarkers for carcinogenesis in some tissues and may provide information regarding the degree of differentiation and aggressiveness of some cancers. Anti-tumor therapy utilizing HSPs are mainly based on two strategies, namely pharmacological modification of HSP expression or molecular chaperone activity, and using them as immunological adjuvants in anti-tumor vaccines.

HSP70 is highly inducible by different stresses, and are either not expressed or expressed in very low levels in normal cells (see e.g., Schmitt et al., 2007; Soti et al., 2005; Calderwood et al,, 2005; Garrido et al., 2006; Ciocca and Calderwood, 2005; and Kregel, 2002). HSP70 can prevent cell death by inhibiting aggregation of cell proteins and directly antagonizing multiple cell death pathways. HSP70 basal level is unusually high in a wide variety of tumors and can be found intracellularly, expressed on plasma membrane or in extracellular medium. HSP70 is correlated with poor prognosis in breast, endometrial, cervical, and bladder carcinomas and is implicated in resistance to chemotherapy in breast cancer. However, HSP70 expression predicts a better response to chemotherapy in osteosarcoma (Ciocca and Calderwood, 2005).

HSP70 family members possess a C terminus domain that chaperones unfolded proteins and peptides, and an N-terminus ATPase domain that controls the opening and closing of the peptide binding domain (Calderwood et al., 2005). HSP70 can form stable complexes with cytoplasmic tumor antigens that can then escape intact from dying cells. HSP70-peptide complexes (HSP70-PC) deliver antigens for re-presentation by MHC class I and II molecules on the antigen-processing cell (APC) surface leading to specific anti-tumor immunity. Therefore, HSP70-PC can potentially break tolerance and cause tumor regression (see e.g., Schmitt et al., 2007; Soti et al., 2005; Calderwood et al., 2005; Garrido et al., 2006; Ciocca and Calderwood, 2005; and Kregel, 2002).

### HSP Induction by Hyperthermia

The kinetics of HSP induction and thermotolerance development have been well studied (see e.g., Schmitt et al., 2007; Soti et al., 2005; Calderwood et al., 2005; Garrido et al., 2006; Ciocca and Calderwood, 2005; Kregel, 2002; and Landry et al., 1982). Using Morris hepatoma 7777 cells heat conditioned at 43° C. for 30 min Landry et al. showed that both HSP induction and thermotolerance development are completed after a 6 to 8 hr period (Landry et al,, 1982). Elevation of HSP70 level was detected immediately after treatment (0-2 hr) and is maximum (more than 4× normal) at 2 hr after thermal stress (Landry et al., 1982). Li et al. found that after an initial treatment at 43° C. for 15 min the rate of synthesis of HSP70 was greatly enhanced in squamous cell carcinomas (SCC VII/SF) when compared to unheated controls (Le and Mak, 1985), The rate of synthesis of HSP70 reached a maximum at 2 to 4 hr after thermal stress and returned to the control rate by 24 hr (Li and Mak, 1985). The response of HSP70 to heat stress was also found to be tissue specific. Flanagan et al. found that hyperthermia induced increase in HSP70 in the liver, small intestine, and kidney, but not in the brain or quadriceps muscles of rats (Flanagan et al., 1995). A higher heat rate (0.166° C./min) was found to be more effective in HSP70 induction as compared to a lower heat rate (0.045° C./min) (Flanagan et al., 1995). HSP70 induction by heat stress can be modulated by a number of factors including nicotine, ethanol, aging, and exercise (Kregel, 2002; Kregel et al., 1995; Kregel and Moseley, 1996; and Hahn et al., 1991).

### MDM2

The tumor-suppressor p53 is a short-lived protein that is maintained at low, often undetectable levels in normal cells. Stabilization of the protein in response to an activating signal, such as DNA damages, results in a rapid rise in p53 level and subsequent inhibition of cell growth. MDM2 binds the p53 tumor-suppressor with high affinity and negatively modulates its transcriptional activity and stability. The MDM2 gene has been found amplified or overexpressed in many human malignancies that effectively impair p53 function (Freedman et al., 1999; and Momand et al., 1998). Therefore, activation of the p53 pathway through inhibition of MDM2 has been proposed as a good therapeutic strategy (Cherie, 2003; and Lane, 1999). Several studies have shown that disruption of the p53-MDM2 interaction by different macromolecular approaches or by the suppression of MDM2 expression can lead to the activation of p53 and tumor growth inhibition (Chen et al., 1998; and Chene et al., 2000). Inhibition with small molecules is a more attractive proposal due to the pharmacological advantages of small molecule drugs, such as enhanced stability and oral bioavailability. Researchers first reported a series of potent and selective small-molecule inhibitors (Nutlins analogs) of the MDM2-p53 interaction with in vitro and in vivo antitumor activity in 2004 (Vassilev et al., 2004). These cis-imidazoline derivatives bind tightly into the p53 pocket of MDM2 and displace p53 from its complexes with MDM2 in vitro with IC₅₀ in the 100-300 nM range. Historically, it has been difficult to develop small-molecule inhibitors of non-enzyme protein-protein interactions because of their large and shallow interfaces. The crystal structure of MDM2-Nutlin complexes revealed that Nutlins project functional groups into the binding pocket that mimic to a high degree the interaction of the three amino acids in p53 critical for this interaction: Leu2, Phe19, and Trp23.

Bifunctional Ligands with Synergistic Tumor Suppressive Functions

A bifunctional ligand, according to the invention, with at least one moiety designed to have good affinity to HSP70 and at least a second moiety designed to inhibit the p53-MDM2 interaction, can potentially have three synergistic tumor suppressive functions. First, selective depletion of HSP70 with this bifunctional ligand can potentially lead to tumor-specific apoptosis (Jaattela, 1999; and Nylandsted et al., 2000). Second, the second moiety designed to inhibit the p53-MDM2 interaction can provide tumor suppressive effects, as discussed above. Third, the bulk of HSP70 may increase the effectiveness of Nutlin in blocking the p53-MDM2 interaction. Gestwicki et al. used this strategy to yield potent inhibitors of β-amyloid (Aβ) aggregation by using bifunctional ligands with a moiety binding to chaperones to increase steric bulk and a moiety available for interaction with Aβ. HSP70 can also be used as a handle controlled by HIFU to localize the MDM2-P53 inhibitor to the tumor site.

### Construction and Design of Bifunctional Molecules

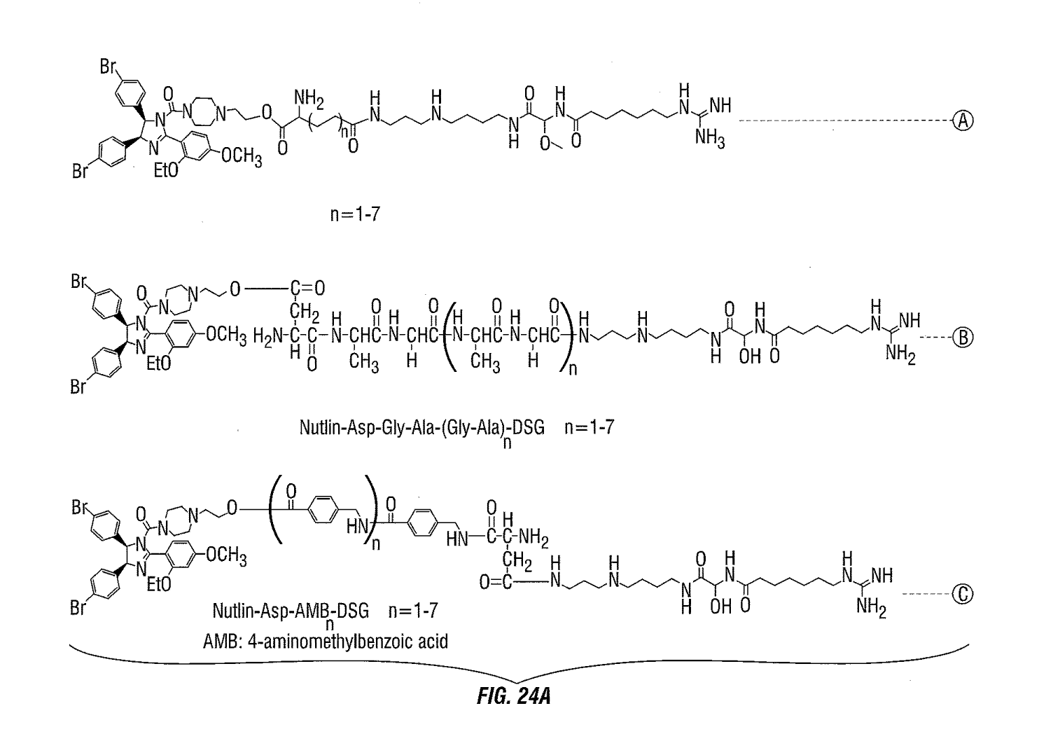

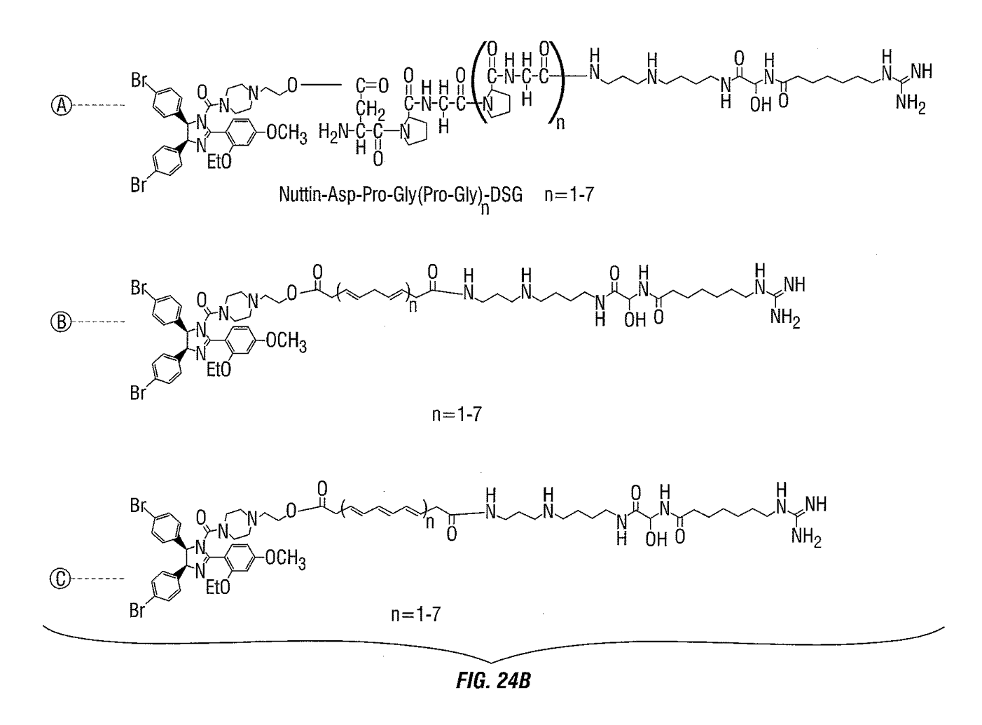

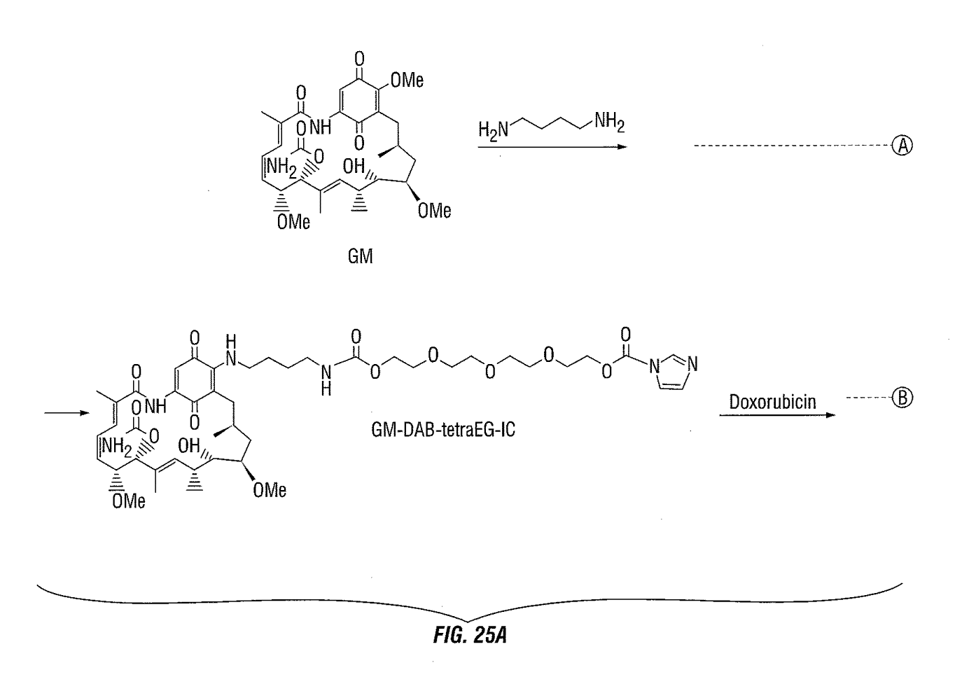

Because the length and flexibility of linker can have a strong impact on the avidity and specificity on bifunctional molecule construction, further development of bifunctional molecule with linkers with different physicochemical properties, different length and flexibility was carried out with computer modeling. As a rational follow on to the original molecule synthesis, the inventors considered a series of different linkers from flexible carbon chains, semirigid Pro-Gly (P-G) and Ala-Gly (A-G) oligomers, to rigid phenyl linkers, 4-aminomethyl benzoic acid (AMB) to construct bifunctional molecule. Compounds with good potential in silico modeling are then synthesized and validated, using standard in vitro and in vivo assays as described elsewhere herein. The Pro-Gly linker was a reasonable choice as it occurs naturally in scaffolds, such as collagen and in silk. Unsaturated acids, like docosahexaenoic acid (DHA, ω3), is healthy and has positive effect on many diseases. In the present design, linkers with structure similar to DHA, which contains multiple double bonds (—C═C—C═; —C═C—C—C═), were also selected for computer modeling. The structure of bifunctional molecules with different linkers is shown in FIG. 24.

Under the modeling study, bifunctional molecule with varying linker lengths and compositions were constructed, and molecular dynamics simulation was performed with CHARMM 35 force field. For the highly flexible ligands, low energy conformations were generated through a simulated annealing (SA) search. This was done by carrying out an MD trajectory at 1500° K. followed by energy minimization under the condition R-die and GB/SA. A set of 10-20 lowest energy conformers, clustered according to coordinate RMS deviations, are docked to the MDM2 and P53 using AutoDock 3.0 rigid-body docking method. Changes between the favored conformations in the free and bound state of the ligand are then used to formulate possible conformational dynamics during binding. Bifunctional molecules with (A-G)ₙ₁ (P-G)ₙ₁ and (AMB)ₙ linker showed a lowering in the conformational energy while with double bond linker (—C═C—C═)ₙ and (C═C—C—C═)ₙ exhibited no substantial changes in the molecules conformational energy (see Table 1). Bifunctional molecules with (A-G)ₙ linker exhibited great decrease conformational energy with n=5, 6, or 7 which predict these structures have great potential for both side binding with MDM2 and HSP70 protein.

TABLE 1

| Linker of | | Conformational |
|---|---|---|
| bifunctional | Linker | Energy |
| molecules | Length | (kcal/mol) |
| (P-G)ₙ | n = 1 | −69 ± 1.5 |
| | n = 2 | −67 ± 1.5 |
| | n = 3 | −83 ± 1.5 |
| | n = 4 | −89 ± 1.5 |
| | n = 5 | −41 ± 1.5 |
| | n = 6 | −80 ± 1.5 |
| | n = 7 | −82 ± 1.5 |
| (A-G)ₙ | n = 1 | very high |
| | n = 2 | −97 ± 1.5 |
| | n = 3 | −125 ± 1.5 |
| | n = 4 | −122 ± 1.5 |
| | n = 5 | −173 ± 1.5 |
| | n = 6 | −193 ± 1.5 |
| | n = 7 | −199 ± 1.5 |
| (AMB)ₙ | n = 1 | −67 ± 1.5 |
| | n = 2 | −79 ± 1.5 |
| | n = 3 | −65 ± 1.5 |
| | n = 4 | −80 ± 1.5 |
| | n = 5 | −74 ± 1.5 |
| | n = 6 | −77 ± 1.5 |
| | n = 7 | −91 ± 1.5 |
| (—C═C—C═)ₙ | n = 1 | −16 ± 1.5 |
| | n = 2 | −6 ± 1.5 |
| | n = 3 | 5 ± 1.5 |
| | n = 4 | 28 ± 1.5 |
| | n = 5 | 39 ± 1.5 |
| | n = 6 | 49 ± 1.5 |
| | n = 7 | 58 ± 1.5 |
| (—C═C—C—C═)ₙ | n = 1 | −17 ± 1.5 |
| | n = 2 | −13 ± 1.5 |
| | n = 3 | −19 ± 1.5 |
| | n = 4 | −2 ± 1.5 |
| | n = 5 | 19 ± 1.5 |
| | n = 6 | 26 ± 1.5 |
| | n = 7 | very high |

### Radionuclides

Exemplary radionuclides useful in the method and compositions of this invention include, but are not limited to, Arsenic-77 (⁷⁷As), Molybdenum-99 (⁹⁹Mo), Rhodium-105 (¹⁰⁵Rh), Lutetium-177 (¹⁷⁷Lu), Cadmium-115 (¹¹⁵Cd), Antimony-122 (¹²²Sb), Promethium-149 (¹⁴⁹Pr), Osmium-193 (¹⁹³Os), Gold-198 (¹⁹⁸Au), Thorium-200 (²⁰⁰Th); preferably Samarium-153 (¹⁵³Sm), Yttrium-90 (⁹⁰Y), Gadolinium-159 (¹⁵⁹Gd), Rhenium-186 (¹⁸⁶Re), Rhenium-188 (¹⁸⁸Re), Holmium-166 (¹⁶⁶Ho), and any combination thereof.

### Cytotoxic Agents and Chemotherapeutics

In some embodiments, the materials of the present invention are provided in combination with existing therapies. In some embodiments, the materials of the present invention are provided to include one or more chemotherapy agents. As such, various classes of antineoplastic (e.g., anticancer) agents are contemplated for use in certain embodiments of the present invention. Anticancer agents suitable for use with the present invention include, but are not limited to, agents that induce apoptosis, agents that inhibit or prevent adenosine deaminase function, inhibit or prevent pyrimidine biosynthesis, inhibit or prevent purine ring biosynthesis, inhibit or prevent nucleotide interconversions, inhibit or prevent ribonucleotide reductase, inhibit or prevent thymidine monophosphate (TMP) synthesis, inhibit or prevent dihydrofolate reduction, inhibit or prevent DNA synthesis, form adducts with DNA, damage DNA, inhibit or prevent DNA repair, intercalate with DNA, deaminate asparagines, inhibit or prevent RNA synthesis, inhibit or prevent protein synthesis or stability, inhibit or prevent microtubule synthesis or function, and the like.

Exemplary anticancer agents suitable for use in compositions and methods of the present invention include, but are not limited to, one or more of those as set forth in U.S. Pat. Nos. 6,979,675 and 7,335,382 (each of which is specifically incorporated herein in its entirety by express reference thereto) inter alia: 1) alkaloids, including microtubule inhibitors (e.g., vincristine [Oncovin®], vinblastine [Velban®], vinorelbine [Navelbine®] and vindesine, etc.), microtubule stabilizers (e.g., paclitaxel [Taxol®, Paxene®] and docetaxel [Taxotere®], etc.); chromatin function inhibitors (including topoisomerase inhibitors, such as epipodophyllotoxins and agents that target topoisomerase I, such as topotecan (Hycamtin®), irinotecan (Camptostar®), and isirinotecan, etc. or agents that target topoisomerase II, such as etoposide, VP-16 (Vepesid®), teniposide (Vumon®), etoposide phosphate (Etopophos®), and the like); 2) covalent DNA-binding agents (alkylating agents), including nitrogen mustards (e.g., mechlorethamine, chlorambucil (Leukeran®), glutfosfamide, cyclophosphamide (Cytoxan®, Neosar®), ifosphamide, and busulfan (Myleran®), etc.), nitrosoureas (e.g., procarbazine [Matulane®], lomustine, CCNU [CeeBU®], carmustine [Gliadel], estramustine [Emcyt®], and semustine, etc.), temozolamide (Temodar®), and other alkylating agents (e.g., dacarbazine (DTIC-Dome®), hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (Adriamycin®, Doxil®, Rubex®), epirubicin (Ellence®), vairubicin (Valstar®), and idarubicin (Idamycin®), etc.), anthracenediones anthracycline analogues, such as mitoxantrone (Novantrone®), etc.), bleomycins, etc., and plicamycin (Mithramycin®), etc.; 4) antimetabolites, including antifolates such as methotrexate, etc.), purine antimetabolites (e.g., mercaptopurine, 6-MP [Purinethol®], fluorouracil, 5-FU [Adrucil®], thioguanine, hydroxyurea [Hydrea®], cytarabine [Cytosar-U®, DepoCyt®], floxuridine, fludarabine [Fludara®], pentostatin [Nipent®], cladribine [Leustatin], gemcitabine [Gemzar®], capecitabine [Xeloda®] acyclovir, ganciclovir, chlorodeoxyadenosine, 2-chlorodeoxyadenosine, and 2′-deoxycoformycin, etc.), pyrimidine antagonists (e.g., fluoropyrimidines [e.g., 5-fluorouracil], 5-fluorodeoxyuridine, etc.), and cytosine arabinosides (e.g., ara-C); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), and aromatase inhibitors (e.g., anastrozole (Arimidex®) and letroazole (Femara®), etc.); 7) platinum compounds (e.g., cisplatin and carboplatin, etc.); 8) monoclonal antibodies conjugated with anticancer drugs, toxins, and/or radionuclides, etc.; 9) biological response modifiers (e.g., interferons [including IFN-α, etc.) and interleukins (e.g., IL-2, etc.), retinoids, including but not limited to, tretinoin, ATRA [Vesanoid®], alitretinoin [Panretin®], and bexarotene [Targretin®], and tyrosine kinase inhibitors, including but not limited to, axitinib [Pfizer], bosutinib [Wyeth], cediranib (Recentin® [AstraZeneca]), dasatinib (Sprycel® [Bristol-Myers Squibb]), erlotinib (Tarceva® [Roche]), gefitinib (Iressa® [AstraZeneca]), imatinib (Gleevec® [Novartis]), lapatinib (Tykerb® [GlaxoSmithKline]), nilotinib (Tasigna® [Novartis]), sorafenib (Nexavar® [Bayer]), sunitinib (Sutent® [Pfizer]), vandetanib (Zactima® [AstraZeneca]), etc., and derivatives or analogs of any of the foregoing, and any combination thereof.

Exemplary chemotherapeutic agents useful in the practice of the present invention also include, but are not limited to, one or more of arsenic trioxide (Trisenox®), zoledronate (Zometa®), tamoxifen (Nolvadex®), fulvestrant (Faslodex®), thiotepa, melphalan (and its analogs, including those as set forth in U.S. Pat. Nos. 3,032,584 and 3,032,585, each of which is specifically incorporated herein in its entirety by express reference threreto), methotrexate, mitoxantrone, estramustine, bleomycin, vinblastine, taxol, taxanes, thalidomide, etoposide, tamoxifen, paclitaxel, vincristine, dexamethasone, busulfan, cyclophosphamide, bischloroethyl nitrosourea, cytosine arabinoside, and derivatives or analogs of any of the foregoing, and any combination thereof.

The term “chemotherapeutic agent” also includes anti-cancer agents, such as toxins, that are targeted to cancer cells by antibodies against cancer cell antigens, including, without limitation, those as described in published PCT Pat. Appl. Publ. Nos. WO/97/00476 and WO/95/10940 (each of which is specifically incorporated herein in its entirety by express reference thereto). The term “chemotherapeutic agent” also includes monoclonal-antibody-based therapies, such as one or more of trastuzumab (Herceptin® [Genentech]); rituximab (Rituxan® [Biogen IDEC]); ofatumumab, zalutumumab, and zanolimumab (Genmab); ertumaxomab (Rexomun® [Fresenius]); tositumomab (Bexxar® [GlaxoSmithKline]); and pantitumumab (Vectibix® [Amgen]), etc., and derivatives or analogs of any of the foregoing, and any combination thereof.

In other embodiments, the compositions of the present invention may be used to deliver one or more agents that act, either directly or indirectly, to inhibit a protein or an enzyme. Exemplary inhibitors include for example, but are not limited to, P13 kinase inhibitors; LY294002; rapamycin; histone deacetylase inhibitors such as RE)-(1S,4S,10S,21R)-7-[(Z)-ethylidene]-4,21-diisopropyl-2-oxa-12,13 -dithia-5,8,20,23-tetraazabicyclo-[8,7,6]-tricos-16-ene-3,6,9,19,22-pentanone (depsipeptide); heat shock protein 90 (Hsp90) inhibitors such as geldanamycin, 17-allylamino-geldanamycin (17-AAG), and other geldanamycin analogs, and radicicol and radicicol derivatives; genistein; indanone; staurosporin; protein kinase-1 (MEK-1) inhibitors such as 2′-amino-3′-methoxyflavone; 1-methylpropyl 2-imidazolyl disulfide; quinoxaline 1,4-dioxides; sodium nitropurruside (SNP) and other NO donors; novobiocin, panzem, 2-methoxyestradiol, epothilone, discodermolide, coumarins; barbituric and thiobarbituric acid analogs; camptothecins; etc., and derivatives or analogs of any of the foregoing, and any combination thereof.

### Anti-Angiogenic Agents

In other embodiments, the compositions of the present invention may be used to deliver, localize, or target one or more anti-angiogenic agents to particular cells or tissues of an animal. Exemplary antiangiogenic agents include, but are not limited to, angiostatin, batimastat, captopril, cartilage derived inhibitor, genistein, endostatin, interleukin, lavendustin A, medroxypregesterone acetate, tecogalan, thalidomide, thrombospondin, Avastin®, Cox-2 inhibitors such as celecoxib (Celebrex®), diclofenac (Voltaren®), etodolac (Lodine®), fenoprofen (Nalfon), indomethacin (Indocin®), ketoprofen (Orudis®), ketoralac (Toradol®), oxaprozin (Daypro®), nabumetone (Relafen®), sulindac (Clinoril®), tolmetin (Tolectin®), ibuprofen, naproxen, aspirin, and acetaminophen, and derivatives or analogs of any of the foregoing, and any combination thereof.

### Antimicrobial Agents

Useful antibiotic agents include, but are not limited to, aminoglycosides, cephalosporins, macrolides, monobactams, penicillins, quinolones, sulfonamides, tetracyclines, 2-isocephem and oxacephem derivatives (see e.g., U.S. Pat. No. 5,919,925); pyridonecarboxylic acid derivatives (see e.g., U.S. Pat. No. 5,910,498); water miscible esters of mono- and diglycerides (see e.g., U.S. Pat. No. 5,908,862); benzamide derivatives (see e.g., U.S. Pat. No. 5,891,890); 6-O-substituted ketolides (see e.g., U.S. Pat. No. 5,866,549); benzopyran phenol derivatives (see e.g., U.S. Pat. No. 5,861,430); pyridine derivatives (see e.g., U.S. Pat. No. 5,859,032); 2-aminothiazole derivatives (see e.g., U.S. Pat. No. 5,856,347); penem ester derivatives (see e.g., U.S. Pat. No. 5,830,889); carbapenem derivates (see e.g., U.S. Pat. No. 5,756,725); N-acylpiperazine derivatives (see e.g., U.S. Pat. No. 5,756,505); oxathiazine oxides (see e.g., U.S. Pat. No. 5,712,275); 5-amidomethyl α, β-saturated and -unsaturated 3-aryl butyolactones (see e.g., U.S. Pat. No. 5,708,169), as well as analogs and derivatives thereof, and any combination thereof. Each of the cited patents is specifically incorporated herein in its entirety by express reference thereto.

### Antifungal Therapeutics

In certain embodiments, the present invention contemplates administration of an antifunal agent using the drug delivery compounds of the present invention. Exemplary antifungal agents include, but are not limited to terpenes, sesquiterpenes diterpenes, and triterpenes (see e.g., U.S. Pat. No. 5,917,084); sulfur-containing heterocyclic compounds (see e.g., U.S. Pat. No. 5,888,526); carbozamides (see e.g., U.S. Pat. No. 5,888,941); phyllosilicates (see e.g., U.S. Pat. No. 5,876,738); corynrcandin derivatives (see e.g., U.S. Pat. No. 5,863,773); sordaridin derivatives disclosed in U.S. Pat. No. 5,854,280); cyclohexapeptides (see e.g., U.S. Pat. No. 5,854,213); terpene compounds (see e.g., U.S. Pat. No. 5,849,956); triazoles (see e.g., U.S. Pat. No. 5,773,443); fusacandins (see e.g., U.S. Pat. No. 5,773,421); terbenzimidazoles (see e.g., U.S. Pat. No. 5,770,617); as well as analogs or derivatives; and/or combinations thereof. Each of the cited patents is specifically incorporated herein in its entirety by express reference thereto.

### Pharmaceutical Formulations

In certain embodiments, the present invention concerns formulation of one or more therapeutic or diagnostic agents in a pharmaceutically-acceptable composition for administration to a cell or an animal, either alone, or in combination with one or more other modalities of prophylaxis and/or therapy. The formulation of pharmaceutically acceptable excipients and carrier solutions is well known to those of ordinary skill in the art, as is the development of suitable dosing and treatment regimens for using the particular compositions described herein in a variety of treatment regimens.

In certain circumstances it will be desirable to deliver the stress-inducible targeted drug delivery compositions disclosed herein in suitably-formulated pharmaceutical vehicles by one or more standard delivery devices, including, without limitation, subcutaneously, intraocularly, intravitreally, parenterally, intravenously, intracerebroventricularly, intramuscularly, intrathecally, orally, intraperitoneally, transdermally, topically, by oral or nasal inhalation, or by direct injection to one or more cells, tissues, or organs. The methods of administration may also include those modalities as described in U.S. Pat. Nos. 5,543,158; 5,641,515, and 5,399,363 (each of which is specifically incorporated herein in its entirety by express reference thereto). Solutions of the active compounds as freebase or pharmacologically acceptable salts may be prepared in sterile water, and may be suitably mixed with one or more surfactants, such as hydroxypropylcellulose. Dispersions may also be prepared in glycerol, liquid polyethylene glycols, oils, or mixtures thereof. Under ordinary conditions of storage and use, these preparations contain a preservative to prevent the growth of microorganisms.

For administration of an injectable aqueous solution, without limitation, the solution may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, a sterile aqueous medium that can be employed will be known to those of ordinary skill in the art in light of the present disclosure. For example, one dosage may be dissolved in 1 mL of isotonic NaCl solution, and either added to 1000 mL of hypodermoclysis fluid or injected at the proposed site of infusion, (see, e.g., “Remington's Pharmaceutical Sciences” 15th Edition, pages 1035-1038 and 1570-1580). Some variation in dosage will necessarily occur depending on the condition of the subject being treated. The person responsible for administration will determine, in any event, the appropriate dose for the individual subject. Moreover, for human administration, preparations should meet sterility, pyrogenicity, and the general safety and purity standards as required by FDA Office of Biologics standards.

Sterile injectable compositions may be prepared by incorporating the disclosed drug delivery vehicles in the required amount in the appropriate solvent with several of the other ingredients enumerated above, as required, followed by filtered sterilization. Generally, dispersions can be prepared by incorporating the selected sterilized active ingredient(s) into a sterile vehicle that contains the basic dispersion medium and the required other ingredients from those enumerated above. The compositions disclosed herein may also be formulated in a neutral or salt form. Pharmaceutically-acceptable salts include the acid addition salts (formed with the free amino groups of the protein), and which are formed with inorganic acids such as, without limitation, hydrochloric or phosphoric acids, or organic acids such as, without limitation, acetic, oxalic, tartaric, mandelic, and the like. Salts formed with the free carboxyl groups can also be derived from inorganic bases such as, without limitation, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation, and in such amount as is effective for the intended application. The formulations are readily administered in a variety of dosage forms such as injectable solutions, topical preparations, oral formulations, including sustain-release capsules, hydrogels, colloids, viscous gels, transdermal reagents, intranasal and inhalation formulations, and the like.

The amount, dosage regimen, formulation, and administration of the compositions disclosed herein will be within the purview of the ordinary-skilled artisan having benefit of the present teaching. It is likely, however, that the administration of a therapeutically-effective, pharmaceutically-effective, prophylactically-effective, or diagnostically-effective amount of the disclosed pharmaceutical compositions may be achieved by a single administration, such as, without limitation, a single injection of a sufficient quantity of the delivered agent to provide the desired benefit to the patient undergoing such a procedure. Alternatively, in some circumstances, it may be desirable to provide multiple, or successive administrations of the stress-inducible targeted drug delivery compositions, either over a relatively short, or even a relatively prolonged period of time, as may be determined by the medical practitioner overseeing the administration of such compositions to the selected individual.

Typically, formulations of one or more active ingredients in the drug delivery formulations disclosed herein will contain an effective amount for the selected therapy or diagnosis. Preferably, the formulation may contain at least about 0.001% of each active ingredient, preferably at least about 0.01% of the active ingredient, although the percentage of the active ingredient(s) may, of course, be varied, and may conveniently be present in amounts from about 0.01 to about 90 weight % or volume %, or from about 0.1 to about 80weight % or volume %, or more preferably, from about 0.2 to about 60 weight % or volume %, based upon the total formulation. Naturally, the amount of active compound(s) in each composition may be prepared in such a way that a suitable dosage will be obtained in any given unit dose of the compound. Factors such as solubility, bioavailability, biological t_{1/2}, route of administration, product shelf life, as well as other pharmacological considerations will be contemplated by one of ordinary skill in the art of preparing such pharmaceutical formulations, and as such, a variety of dosages and treatment regimens may be desirable. Preferably, the bifunctional compositions of the present invention may be administered in the same dosage amount as a unifunctional or unfunctionalized pharmaceutical compositions while inhibiting or avoiding one or more adverse effects of such unifunctional or unfunctionalized composition.

The pharmaceutical compositions disclosed herein may be administered by any effective method, including, without limitation, by parenteral, intravenous, intramuscular, or even intraperitoneal administration as described, for example, in U.S. Pat. Nos. 5,543,158, 5,641,515 and 5,399,363 (each of which is specifically incorporated herein in its entirety by express reference thereto). Solutions of the active compounds as free-base or pharmacologically acceptable salts may be prepared in water suitably mixed with a surfactant, such as hydroxypropylcellulose, or other similar fashion. The pharmaceutical forms adapted for injectable administration include sterile aqueous solutions or dispersions, and sterile powders for the extemporaneous preparation of sterile injectable solutions or dispersions including without limitation those described in U.S. Pat. No. 5,466,468, which is specifically incorporated herein in its entirety by express reference thereto. In all cases, the form must be sterile and must be fluid to the extent that easy syringability exists. It must be at least sufficiently stable under the conditions of manufacture and storage, and must be preserved against the contaminating action of microorganisms, such as viruses, bacteria, fungi, and such like.

The carrier(s) can be a solvent or dispersion medium including, without limitation, water, ethanol, polyol (e.g., glycerol, propylene glycol, and liquid polyethylene glycol, and the like, or a combination thereof), one or more vegetable oils, or any combination thereof, although additional pharmaceutically-acceptable components may be included,

Proper fluidity of the pharmaceutical formulations disclosed herein may be maintained, for example, by the use of a coating, such as e.g., a lecithin, by the maintenance of the required particle size in the case of dispersion, by the use of a surfactant, or any combination of these techniques. The inhibition or prevention of the action of microorganisms can be brought about by one or more antibacterial or antifungal agents, for example, without limitation, a paraben, chlorobutanol, phenol, sorbic acid, thimerosal, or the like. In many cases, it will be preferable to include an isotonic agent, for example, without limitation, one or more sugars or sodium chloride, or any combination thereof. Prolonged absorption of the injectable compositions can be brought about by the use in the compositions of agents delaying absorption, for example without limitation, aluminum monostearate, gelatin, or a combination thereof.

While systemic administration is contemplated to be effective in many embodiments of the invention, it is also contemplated that formulations of the disclosed drug delivery compositions may be suitable for direct injection into one or more organs, tissues, or cell types in the body. Such injection sites include, without limitation, the circulatory system, the spinal cord, the lymphatic system, a joint or joint capsule, a synovium or subsynovium tissue, tendons, ligaments, cartilages, bone, periarticular muscle or an articular space of a mammalian joint, as well as direct administration to an organ or tissue site such as the heart, liver, lung, pancreas, intestine, brain, bladder, kidney, or other site within the patient's body, including, for example, without limitation, introduction of the delivered therapeutic or diagnostic agent(s) via intra-abdominal, intra-thoracic, intravascular, or intracerebroventricular delivery of a suitable liposomal formulation. Administration of the disclosed compositions need not be restricted to one or more of these delivery means, but instead may be conducted using suitable means, including those known to the one of ordinary skill in the relevant medical arts. In certain embodiments, the active ingredients of the invention may be formulated for delivery by needle, catheter, and related means, or alternatively, may be included within a medical device, including, without limitation, drug-eluting implants, stents, catheters, and such like. The formulations may also be prepared for injection by an implanted drug-delivery pump or similar mechanism.

The administration of the pharmaceutical compositions disclosed herein may be conducted using any method as conventionally employed in the medical arts, and may include, without limitation, administration of intranasal sprays, inhalation, and/or other aerosol delivery vehicles (see e.g., U.S. Pat. Nos. 5,756,353 and 5,804,212, each of which is specifically incorporated herein in its entirety by express reference thereto). Delivery of drugs using intranasal microparticle resins (see e,g., Takenaga et al., 1998) and lysophosphatidyl-glycerol compounds (U.S. Pat. No. 5,725,871, specifically incorporated herein in its entirety by express reference thereto) are also well-known to those of ordinary skill in the pharmaceutical arts, and may also be employed in the practice of the present methods. Transmucosal drug delivery is also contemplated to be useful in the practice of the invention. Exemplary methods are described, for example, without limitation, in U.S. Pat. No. 5,780,045, which is specifically incorporated herein in its entirety by express reference thereto.

The disclosed pharmaceutical formulations may also be administered through transdermal or other topical administration routes. Exemplary methods for the use of liposomal formulations in topical therapy are found, for example, in U.S. Pat. Nos. 5,540,936, and 6,133,451 (each of which is specifically incorporated herein in its entirety by express reference thereto).

In particular embodiments, the disclosed pharmaceutical compositions may be formulated using one or more pharmaceutical buffers, vehicles, or diluents, and intended for administration to a mammal through a suitable route, such as, by intramuscular, intravenous, subcutaneous, intrathecal, intra-abdominal, intravascular, intra-articular, or alternatively, by direct injection to one or more cells, tissues, or organs of such a mammal

The pharmaceutical formulations disclosed herein are not in any way limited to use only in humans, or even to primates, or mammals. In certain embodiments, the methods and compositions disclosed herein may be employed using avian, amphibian, reptilian, or other animal species.

In preferred embodiments, however, the compositions of the present invention are preferably formulated for administration to a mammal, and in particular, to humans, in a variety of diagnostic, therapeutic, and/or prophylactic regimens. The compositions disclosed herein may also be provided in formulations that are acceptable for veterinary administration, including, without limitation, to selected livestock, exotic or domesticated animals, companion animals (including pets and such like), non-human primates, as well as zoological or otherwise captive specimens, and such like.

Such methods may also encompass prophylactic treatment of one or more animals suspected of having, or at risk for developing one or more such conditions either following diagnosis, or prior to the onset of symptoms. To that end, in certain embodiments the pharmaceutical compositions disclosed and/or described herein may also find utility in the area of vaccine development, and antigen administration/vaccination and the like.

### Definitions

As used herein, the term “carrier” is intended to include any solvent(s), dispersion medium, coating(s), diluent(s), buffer(s), isotonic agent(s), solution(s), suspension(s), colloid(s), inert(s) or such like, or a combination thereof, that is pharmaceutically acceptable for administration to the relevant animal. The use of one or more delivery vehicles for chemical compounds in general, and peptides and epitopes in particular, is well known to those of ordinary skill in the pharmaceutical arts. Except insofar as any conventional media or agent is incompatible with the active ingredient, its use in the diagnostic, prophylactic, and therapeutic compositions is contemplated. One or more supplementary active ingredient(s) may also be incorporated into, or administered in association with, one or more of the disclosed immunogenic compositions.

As used herein, the term “expression” refers to the biological production of a product encoded by a coding sequence. In most cases, a polynucleotide (i.e., DNA) sequence, including the coding sequence, is transcribed to form a messenger-RNA (mRNA). The messenger-RNA is then translated to form a polypeptide product that has a relevant biological activity. The process of expression may involve further processing steps to the RNA product of transcription, such as splicing to remove introns, and/or post-translational processing of a polypeptide product.

As used herein, a “heterologous” is defined in relation to a predetermined referenced gene sequence. For example, with respect to a structural gene sequence, a heterologous promoter is defined as a promoter that does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, the term “operably linked” refers to a linkage of two or more polynucleotides or two or more nucleic acid sequences in a functional relationship. A nucleic acid is “operably linked” when it is placed into a functional relationship with another nucleic acid sequence. For instance, a promoter or enhancer is operably linked to a coding sequence if it affects the transcription of the coding sequence. “Operably linked” means that the nucleic acid sequences being linked are typically contiguous, or substantially contiguous, and, where necessary to join two protein coding regions, contiguous and in reading frame. Since enhancers generally function when separated from the promoter by several kilobases and intronic sequences may be of variable lengths; however, some polynucleotide elements may be operably linked but not contiguous.

The phrases “isolated” or “biologically pure” refer to material that is substantially, or essentially, free from components that normally accompany the material as it is found in its native state. Thus, isolated peptides in accordance with the invention preferably do not contain materials normally associated with the peptides in their in situ environment.

“Link” or “join” refers to any method known in the art for functionally connecting two or more molecules, including, without limitation, recombinant fusion, covalent bonding, disulfide bonding, ionic bonding, hydrogen bonding, electrostatic bonding, and such like.

As used herein, the term “monoclonal,” when used in reference to an antibody, refers to an antibody that is based upon, obtained from or derived from a single clone, including any eukaryotic, prokaryotic, or phage clone. The term monoclonal antibody is often abbreviated “MAb” in the singular, and “MAbs” in the plural.

As used herein, the term “patient” (also interchangeably referred to as “host” or “subject”) refers to any host that can receive one or more of the pharmaceutical compositions disclosed herein. Preferably, the subject is a vertebrate animal, which is intended to denote any animal species (and preferably, a mammalian species such as a human being). In certain embodiments, a “patient” refers to any mammalian host, including but not limited to, human and non-human primates, bovines, canines, caprines, cavines, corvines, epines, equines, felines, hircines, lapines, leporines, lupines, murines, ovines, porcines, ranines, racines, vulpines, and the like, including livestock, zoological specimens, exotics, as well as companion animals, pets, and any animal under the care of a veterinary practitioner. A patient can be of any age at which the patient is able to respond to inoculation with the present vaccine by generating an immune response. In particular embodiments, the mammalian patient is preferably human.

The phrase “pharmaceutically-acceptable” refers to molecular entities and compositions that preferably do not produce an allergic or similar untoward reaction when administered to a mammal, and in particular, when administered to a human. As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of or interchangeably with, any of these terms. The term further includes polypeptides that have undergone one or more post-translational modification(s), including, without limitation, glycosylation, acetylation, phosphorylation, amidation, derivatization, proteolytic cleavage, post-translation processing, or modification by inclusion of one or more non-naturally occurring amino acids. Throughout the, disclosure, common one-letter and three-letter amino acid abbreviations have been employed following the conventional nomenclature in the art: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (1; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “L” isomeric form. However, residues in the “D” isomeric form may be substituted for any L-amino acid residue provided the desired properties of the polypeptide are retained. All amino-acid residue sequences represented herein conform to the conventional left-to-right amino-terminus to carboxy-terminus orientation.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about 2 to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. Polypeptides and proteins of the present invention also include polypeptides and proteins that are or have been post translationally modified, and include any sugar or other derivative(s) or conjugate(s) added to the backbone amino acid chain.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

As used herein, the term “substantially homologous” encompasses sequences that are similar to the identified sequences, such that antibodies raised against peptides having the identified sequences will react with peptides having the substantially homologous sequences. In some variations, the amount of detectable antibodies induced by the homologous sequence is identical to the amount of detectable antibodies induced by the identified sequence. In other variations, the amounts of detectable antibodies induced are substantially similar, thereby providing immunogenic properties. For example, “substantially homologous” can refer to at least about 75%, preferably at least about 80%, and more preferably at least about 85% or at least about 90% identity, and even more preferably at least about 95%, more preferably at least about 97% identical, more preferably at least about 98% identical, more preferably at least about 99% identical, and even more preferably still, at least substantially or entirely 100% identical (i.e., “invariant”). [00147]As used herein, the terms “treatment,” “treat,” “treated,” or “treating” refer to therapy, or to the amelioration or the reduction, in the extent or severity of disease, or a symptom thereof, whether before or after its development afflicts a patient. When used with respect to an infectious disease, for example, the terms refer to a treatment or treatment regimen that decreases the severity of the infection or decreases or lessens or delays one or more symptoms of illness attributable to the infection, as well as increasing the ability of the infected individual to fight the infection, including e.g., the reduction and/or elimination of the infection from the body of the individual, or to lessen or prevent the disease from becoming worse.

The term “for example” or “e.g.,” as used herein, is used merely by way of example, without limitation intended, and should not be construed as referring only those items explicitly enumerated in the specification.

In accordance with long standing patent law convention, the words “a” and “an” when used in this application, including the claims, denote “one or more.”

### EXAMPLES

The following examples are included to demonstrate illustrative embodiments of the invention. It should be appreciated by those of ordinary skill in the art that the techniques disclosed in the examples that follow represent techniques discovered by the inventor to function well in the practice of the invention, and thus can be considered to constitute preferred modes for its practice. However, those of ordinary skill in the art should, in light of the present disclosure, appreciate that many changes can be made in the specific embodiments that are disclosed and still obtain a like or similar result without departing from the spirit and scope of the invention.

### Example 1

### Novel Imaging Probes for the Detection of a Heat-Inducible Molecular Target

In an effort to develop a probe capable of detecting HSP70 in vivo two fluorescent derivatives of (−)-15-DSG have been synthesized by the coupling 6-carboxyfluorescein-N-hydroxysuccinimide ester (DSG-FAM) and Cy5.5 monoester (DSG-Cy5.5). 15-DSG consists of an unstable α-hydroxyglycine central part, connecting two highly-polar moieties: guanidinoheptanoic acid and spermidine. Owing to the unusual hemiaminal structure of the α-hydroxyglycine unit, DSG hydrolyses gradually, in basic or acidic aqueous solution, into 7-guanidinoheptanamide and hydrated glyoxylspermidine. This example describes a solution for the significant challenge of synthesizing and purifying this hygroscopic unstable salt derivative in sufficient quantity.

### Synthesis of 15-Deoxyspergualin (15-DSG)

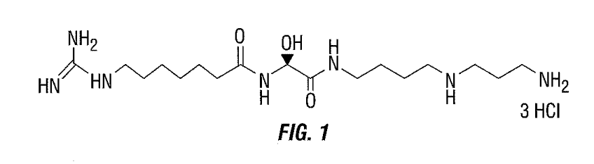

15-deoxyspergualin (15-DSG) (FIG. 1) is a promising antitumor and immunosuppressive antibiotic agent, that is known to bind to HSP70. 15-DSG has been found to be more effective than popular immunosuppressants such as like Cyclosporin A, FK 506, or Rapamycin, and routinely elicits fewer side effects and a different mechanism of action. (±)15-DSG has a rather unstable peptidomimetic structure containing an asymmetric carbon. Although in vivo studies clearly showed that (±)15-DSG is the only immunosuppressive enantiomer and that both isomers contribute at least to the acute toxicity, most of the biological, pharmacological and clinical data have been obtained with the more readily available racemic tris-hydrochloride deoxyspergualin. Recently it was marketed in Japan for the control of corticoresistant acute renal graft rejection, but it is apparently not yet commercially available.

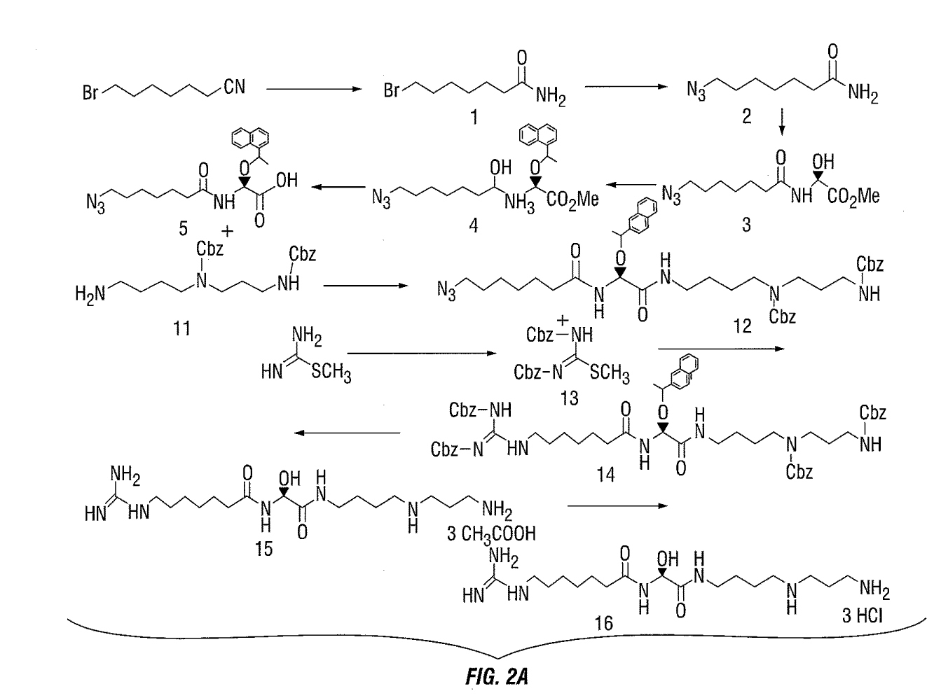

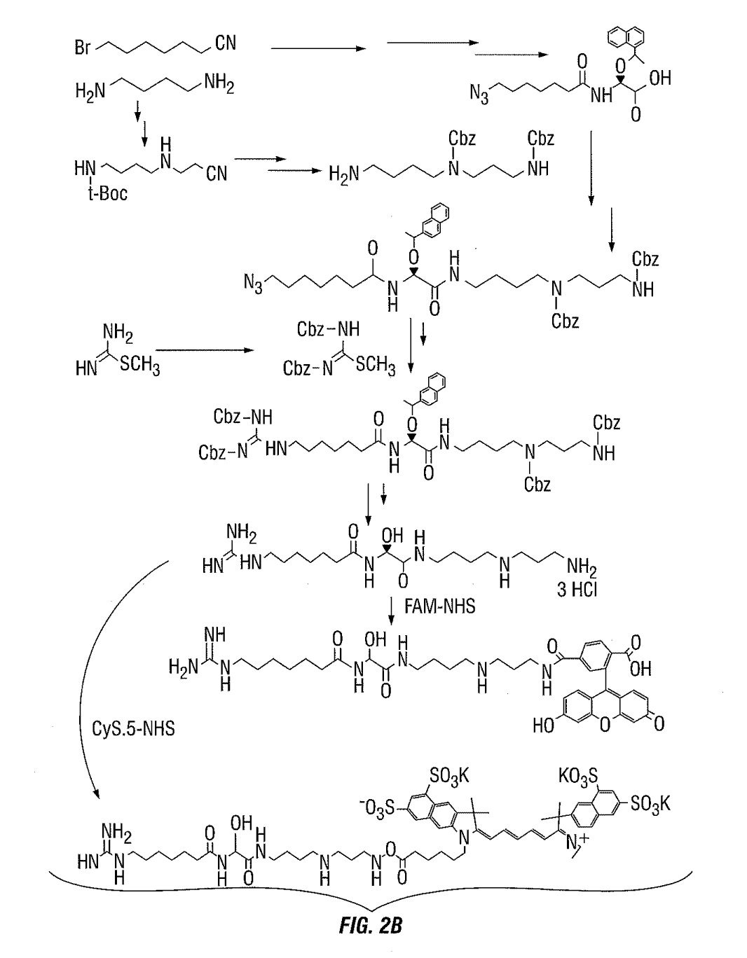

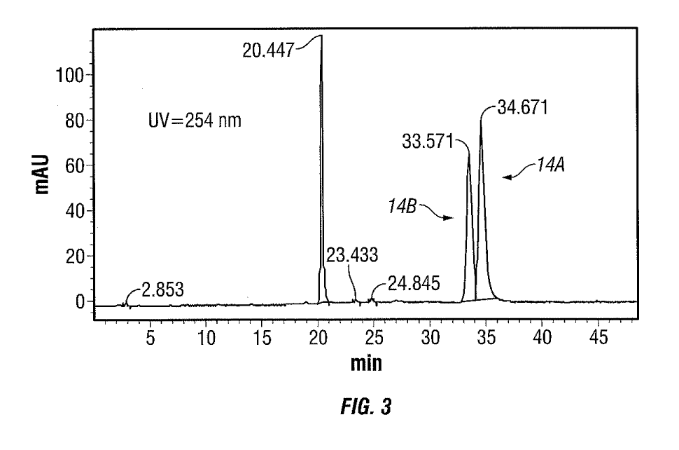

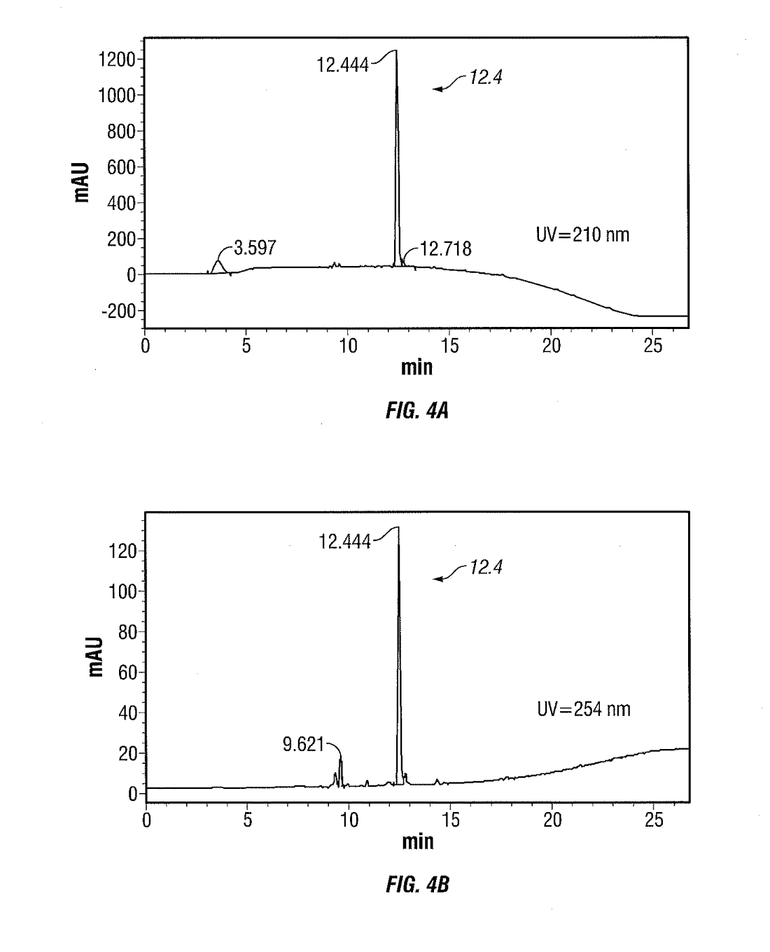

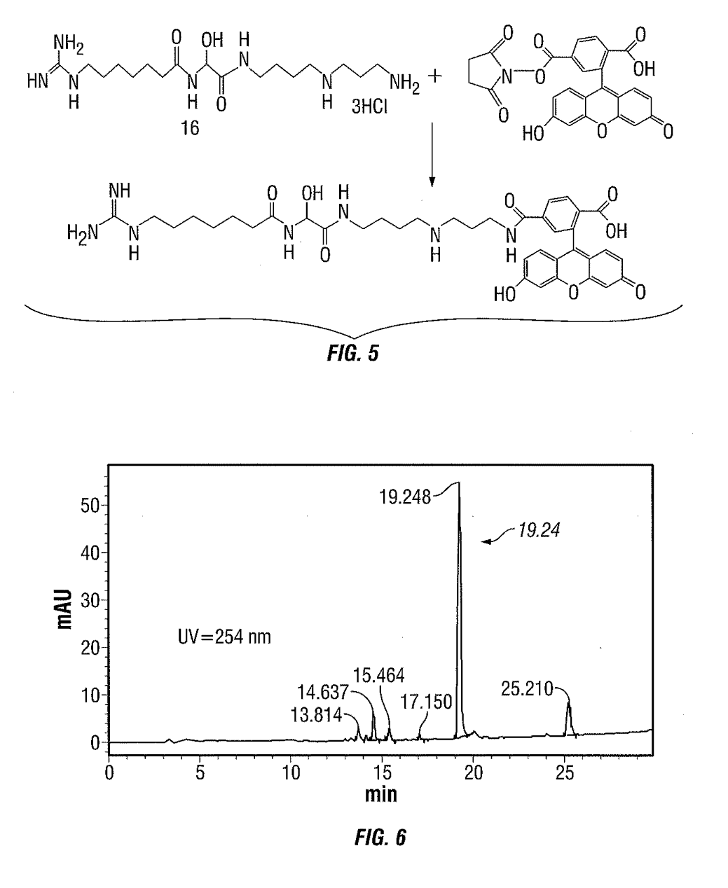

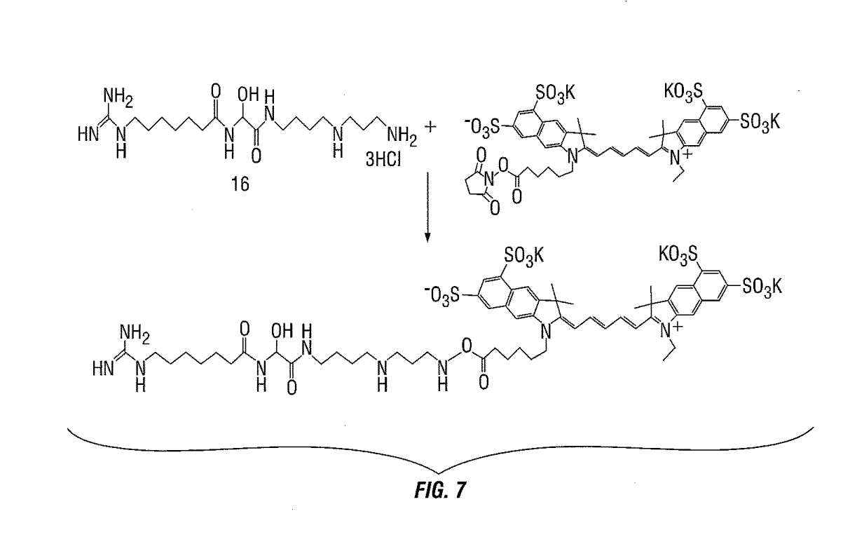

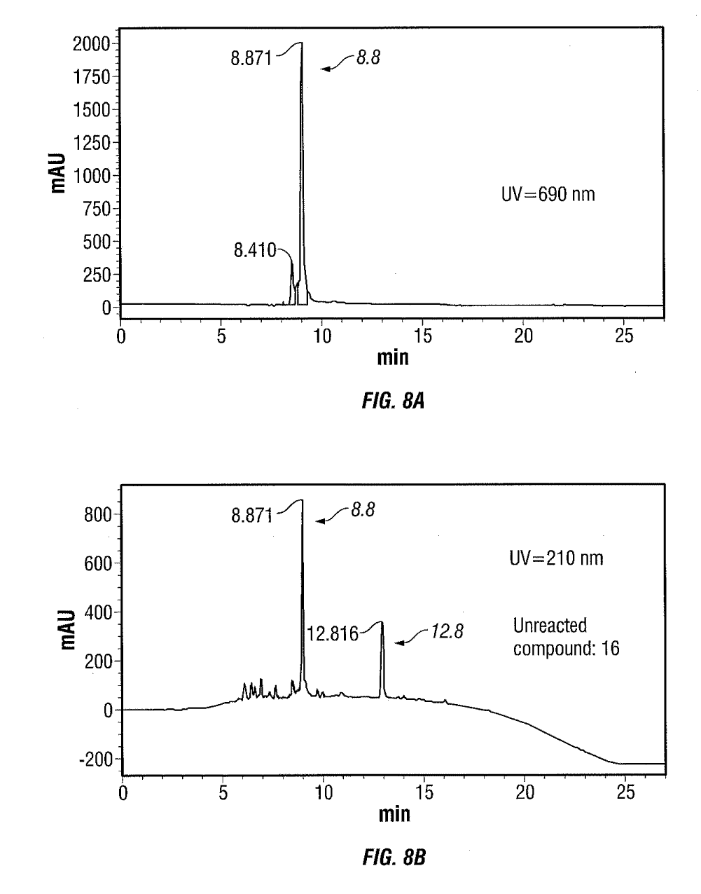

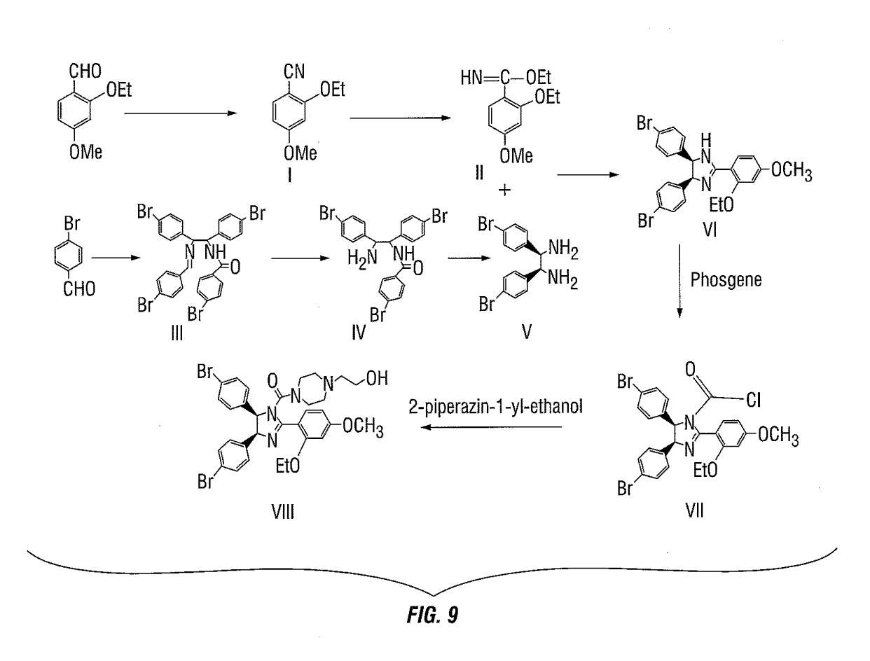

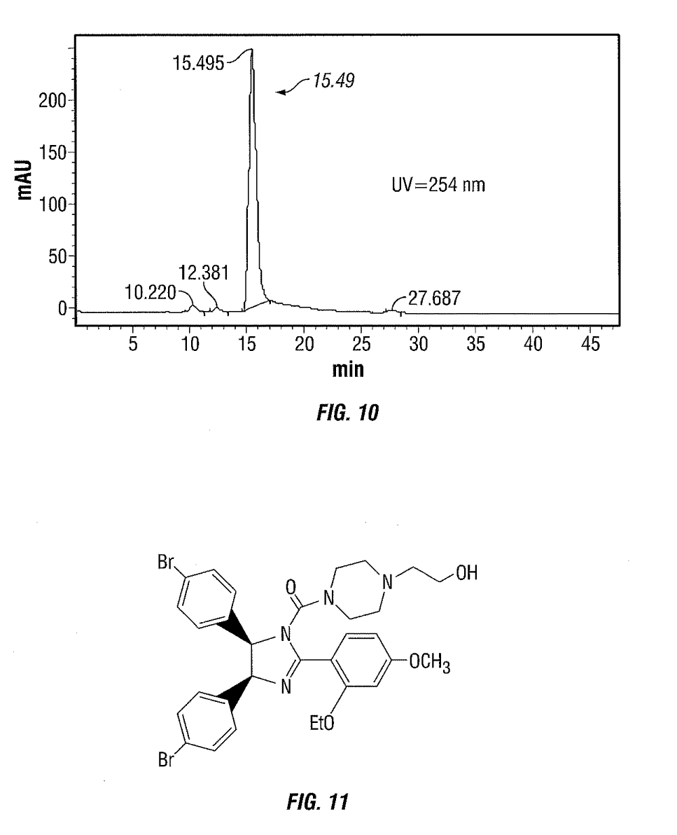

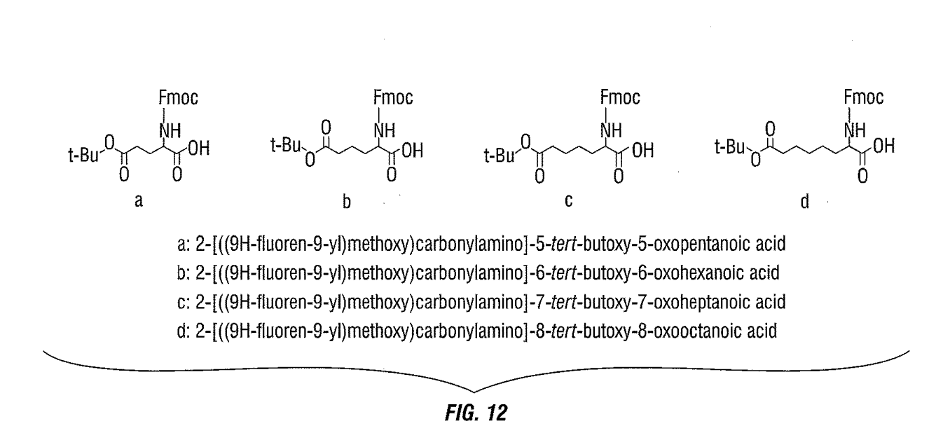

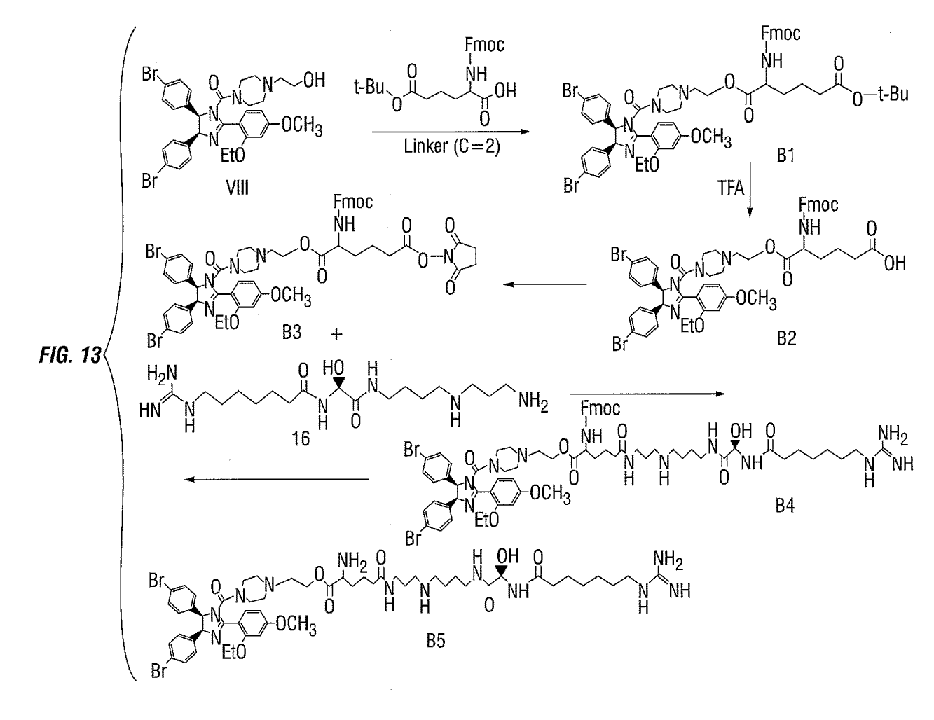

Efficient synthesis of (±)7-[(aminoiminomethyl)amino]-N-[2-[[4-[(3-aminopropyl)amino]butyl]amino]-1-hydroxy-2-oxoethyl]heptanamide trihydrochloride, [(±)-15-DSG], has been developed starting from 7-bromoheptanenitrile, protected spermidine N¹,N⁴-bis(tertbutoxycarbonyl)spermidine and guanidine reagent N,N′-bis(tert-butoxycarbonyl)-S-methylisothiourea, suitable for the production of multi-gram quantities of this unstable highly polar compound with an 25-30% overall yield (FIG. 2A and FIG. 2B). The chemical purity of the final product was determined to be >98% by HPLC analysis.

The product was then further purified and transformed to the tris(hydrochloride) following a protocol on CM C-25 Sephadex and Sephadex LH-20 columns. The collected fractions were lyophilized and freeze-dried product was obtained as a hygroscopic powder. The chemical purity was again determined by an analytical HPLC system (1200 series pump, Agilent, Germany) using a Vydac protein and peptide C₁₈ column.

The pure 15-DSG was dissolved in DMF with triethylamine followed by the addition of 6-carboxylfluorescein N-hydroxysuccinimide ester or Cy5.5 mono NHS ester and incubated overnight at 40C to produce DSG-FAM or DSG-Cy5.5 respectively. These fluorescent agents were purified on a semi preparative HPLC system through a Luna SCX 100A column. The details of the synthesis are provided below:

### Synthesis of Compound 1

7-bromoheptanenitrile (3.0 gm, 15.78 mmol) was dissolved in 75 mL of concentrated hydrochloric acid (d 1.19) and stirred for 20 hr at room temperature (advantageously at 15-20° C.). This mixture is then poured onto ice-cold water (200 mL) and the white precipitate obtained is then filtered off. After washing with water and evaporated to dryness. The crude product is re-crystallized from ethyl acetate-methylcyclohexane solvent mixture to give 2.67 gm of the expected product 1 in the form of white crystals (81% yield).

MS: M+1 calculated 209.09, found 209.26.

### Synthesis of Compound 2

Compound 1 (2.5 gm, 12.01 mmol) was dissolved in dimethylsulfoxide (DMSO) (30 mL) in a dry flask under argon and sodium azide (1.56 gm, 24.03 mmol) was added. The reaction mixture was stirred for 4 h at 90° C. when thin-layer chromatography (TLC) showed that no starting material remained. The cooled mixture was dissolved in ethyl acetate (150 mL) and washed with water (3×150 mL). The organic phase was dried (MgSO₄), evaporated to dryness and the crude product was purified on a silica gel column and using 5% isopropanol in ethyl acetate as eluent. This afforded product 2 as a white crystalline solid (1.5 gm, 74% yield).

MS: M+1 calculated 171.21, found 171.39.

### Synthesis of Compound 3

Compound 2 (1.0 gm, 5.88 mmol) was dissolved in dichloromethane (40 mL) in a flask connected to a Soxhlet apparatus filled with about 10 gm of 4 Å molecular sieves under Argon. 2-hydroxy-2-methoxy acetate (0.70 mL, 7.05 mmol) was added and the reaction mixture heated under reflux with stirring for 30 hr, when TLC showed that no significant starting material remained. The mixture was concentrated under reduced pressure and the residue was dissolved in dichloromethane (60 mL). The solution was washed with water (3×60 mL). The organic phase was dried (MgSO₄), evaporated to dryness and purified by flash chromatography on silica gel column using 2% methanol in dichloromethane. The pure compound 3 (1.2 gm) was obtained as a solid in 70% yield.

MS: M+1 calculated 259.27, found 259.48.

### Synthesis of Compound 4

Compound 3 (1.0 gm, 3.87 mmol) was dissolved in dichloromethane (20 mL) in a dry flask under argon atmosphere and thionyl chloride (0.4 mL, 5.42 mmol) was added drop wise into the mixture. The resulting mixture was refluxed at 50° C. for 2 hr, when TLC showed that no starting material remained. The reaction mixture was concentrated under reduced pressure. The crude chloroglycine derivative was dissolved in 20 mL of dichloromethane, (S)-(−)-R-methyl-2-naphthalenemethanol (0.73 gm, 4.26 mmol) was added drop wise, followed by addition of triethylamine (1.08 mL, 7.74 mmol) in 10 mL of dichloromethane. The reaction mixture was stirred at room temperature for 40 hr, when TLC showed that no significant starting material remained. The reaction mixture was concentrated and dissolved in dichloromethane (100 mL), washed with 1 (N) HCl (60 mL) and brine (60 mL). The organic layer was dried (MgSO₄), concentrated and the crude residue was purified by a silica gel column using 1% isopropanol in hexane as the eluent to give 1.2 gm (75%) of a mixture of diastereomeric esters, which was directly used in the next step.

MS: M+1 calculated 432.52, found 432.66.

### Synthesis of Compound 5

Compound 4 (2.8 gm, 6.8 mmol) was taken in a round bottom flask and dissolved in 1,2-dimethoxyethane (40 mL). 1(N) sodium hydroxide (8.2 mL, 8.15 mmol) solution was added to the above solution and starred for 2 hr at room temperature, when TLC showed that no starting material remained. The solvent was evaporated and the residue was diluted with water (100 mL) and acidified with 1 (N) HCl to pH=2. The aqueous phase was extracted with ethyl acetate (3×100) and the organic phase was dried (MgSO₄) and concentrated under reduced pressure to yield 2.6 gm (96%) of the crude diastereomeric mixture of acids in the form of oil.

MS: M+1 calculated 399.45, found 399.71.

### Synthesis of Compound 12

Compound 5 (2.5 gm, 6.27 mmol) was dissolved in dichloromethane (60 mL) in a dry flask under argon and hydroxybenzotriazole (0.85 gm, 6,27 mmol) was added, followed by addition of N,N′dicyclohexylcarbodiimide (1.42 gm, 6.9 mmol). The reaction mixture stirred for 2 hr at room temperature and N¹,N⁴-bis(benzyloxycarbonyl)spermidine, Compound 11 (2.85 gm, 6.9 mmol) was added. The reaction mixture was further stirred for 40 hr at room temperature when TLC showed no starting material remained. Solvent was evaporated under vacuum, the residue was dissolved in dichloromethane (100 mL) and washed with saturated aqueous sodium bicarbonate (3×100 mL). The organic phase was dried (MgSO₄), evaporated to dryness and the crude product was purified on a silica gel column using 3% isopropanol in ethyl acetate as the eluent to give 4.1 gm (82%) of a mixture of epimers, which was directly used in the next step.

MS: M+1 calculated 794.95, found 795.18.

### Synthesis of Compound 13

Methyl carbamimidothioate (3.0 gm, 33.28 mmol) was dissolved in tetrahydrofuran (100 mL) under argon and triethylamine (14 mL, 99.84 mmol) was added. The reaction mixture was cooled to 0° C. and then benzyloxycarbonyl chloride (14.19 gm, 83.20 mmol) in tetrahydrofuran (30 mL) was added drop wise over a period of 2 hr. The reaction mixture was warmed to room temperature and stirred overnight when TLC showed that no starting material remained. A precipitate was removed by filtration and the solvent was evaporated to dryness under vacuum. The residue was purified on a silica gel column using 5% methanol in dichloromethane as the eluent. The pure compound 13, N,N′-bis(benzyloxycarbonyl)-S-methylisothiourea, 8.5 gm was obtained in 71% yield.

MS: M+1 calculated 359.41, found 359.77.

### Synthesis of Compounds 14A and 14B

Compound 12 (3.6 gm, 4.53 mmol) was dissolved in tetrahydrofuran (45 mL) and water (5 mL) in a pear-shaped flask. Triphenylphosphine (1.19 gm, 4.53 mmol) was added and the reaction mixture heated with stirring at 70° C. for 20 hr. After cooling the reaction mixture, compound 13 (1.79 gm, 5.0 mmol) was added, and stirred overnight at room temperature after which TLC revealed no significant starting material remained. The solvent was then evaporated, and the residue was purified on a silica gel column eluting with 3% isopropanol in ethyl acetate to produce a mixture of epimers 14 (4.1 gm) in 84% yield. The mixture of epimers was separated by a semipreparative high-performance liquid chromatography (HPLC) system. The separation was performed on an Econosil® C₁₈ column (10 μm, 250×10 mm). The flow rate was 4 mL/min, with the mobile phase starting from 50% solvent A (water) and 50% solvent B (acetonitrile) to 20% solvent A and 80% solvent B over 45 min to produce 14a (2.0 gm, 41%) and 14b (1.8 gm, 37%) as an oil.

MS: M+1 calculated 1079.25, found 1079.47.

### Synthesis of Compound 15

Compound 14a (1.0 gm, 0.93 mmol) was dissolved in 1 (N) acetic acid in methanol (100 mL) under nitrogen atmosphere. To this solution 0.5 gm (50 wt %) of palladium hydroxide (Pearlman's catalyst, 20% on carbon/50% water) was added and stirred for 12 hr under 1 atm of hydrogen at room temperature. The mixture was filtered. In the filtrate, 0.5 gm (50 wt %) of Pearlman's catalyst was added again and the mixture was treated as above overnight. The reaction mixture was filtered and concentrated under reduced pressure. The residue was dissolved in water (50 mL) and washed with dichloromethane (3×50 mL) The aqueous phase was lyophilized. The resulting powder was dissolved in water (20 mL) and lyophilized again to give the triacetate 15 as a hygroscopic white powder (0.27 gm, 75%). The chemical purity was determined by an analytical HPLC system. Analysis was performed on an Inertsil® OSD 2 column (5 μm, 4.6×250 mm). The mobile phase was starting from Solvent A, water with 0.05% trifluoroacetic acid; Solvent B, CH₃CN with 0.05% trifluoroacetic acid: 5% Solvent B in 8 min and 5-80% Solvent B in 25 min Flow rate was 1 mL/min, temperature 31° C.

MS: M+1 calculated 388.52, found 388.64.

### Synthesis of Compound 16

The product 15 was then further transformed to the tris(hydrochloride) following the method. First, CM-Sephadex C-25 (3.0 gm) was equilibrated with water, eluting with water and 0.2 (N), 0.4 (N), 0.6 (N), 0.8 (N) and then 1.0 (N) aqueous sodium chloride (30 mL each). The fractions (ninhydrin active) were collected, combined and lyophilized, stirred with methanol, and filtered. Next a column of Sephadex LH-20 (6.0 gm) was pre-swelled with methanol and also elution with methanol. The collected fractions were lyophilized. The freeze-dried product was obtained as a hygroscopic powder (0.26 gm, 73%). The chemical purity was determined by an analytical HPLC system. The quality control analysis was performed on Grace Vydac protein and peptide C₁₈ column (5 μm, 150×4.6 mm). The mobile phase started from 95% Solvent A (0.1% trifluoroacetic acid in water) and 5% Solvent B (0.1% trifluoroacetic acid in acetonitrile; 0 to 3 min) to 20% Solvent A and 80% Solvent B for 25 min. Flow rate was 1 mL/min, temperature 31° C.

¹H NMR 16 (D₂O) δ: 5.36 (s, 1H, CH, methine), 3,21 (m, 4H, CH₂, methylene), 3.02 (m, 6H, CH₂, methylene), 2.21 (t, 2H, J=7.5 Hz, CH₂, methylene), 2.02 (m, 2H, CH₂, methylene), 1.54 (m, 8H, CH₂, methylene), 1.28 (m, 4H, CH₂, methylene). ¹³C NMR 16 (D₂O) δ: 175.02, 170.67, 154.39, 71.43, 49.09, 46.16, 41.81, 40.21, 37.08, 36.28, 29.07, 28.42, 26.92, 26.00, 25.67, 25.06, 22.23.

MS: HRMS (M+1): calculated 388.2508, found 388.3041.

### Example 2

### Synthesis of Fluorescent DSG

### Synthesis of FAM-DSG

Compound 16, 15-DSG (50 mg, 0.13 mmol) was dissolved in N,N-dimethylformamide (DMF) (0.1 mL) under argon and triethylamine (65 μL, 0.39 mmol) was added. The reaction mixture was cooled to 0° C. and then 6-carboxyfluorescein (FAM) N-hydroxysuccinimide ester (123 mg, 0.26 mmol) in N,N-dimethylformamide (DMF) (30 μL) was added. The reaction mixture was warmed to room temperature and stirred overnight. The solvent was evaporated to dryness under vacuum. The purification of the crude product was carried out on a semipreparative HPLC system. Purification was performed on a Luna SCX 100A column (5 μm, 250×10 mm) The flow was 4 mL/min, with the mobile phase starting from 95% solvent A (0.1% trifluoroacetic acid in water) and 5% solvent B (0.1% trifluoroacetic acid in acetonitrile; 0 to 3 min) to 20% solvent A and 80% solvent B at 30 min.

The peak containing color desired product was collected, dried and stored in the dark at −20° C. until use. The pure compound, FAM-DSG, 65 mg was obtained in 67% yield. The quality control analysis was performed using the same gradient system described above with a Vydac, protein and peptide C₁₈ column (5 μm, 150×4.6 mm) and flow was 1 mL/min

¹H NMR 16 (CD₃OD) δ: 8.47 (t, 2H, J=8.1 Hz, amine), 8.35 (m, 3H, amine and amide), 8.16 (m, 1H, amide), 7.68 (s, 1H, aromatic), 6.85 (m, 4H, aromatic), 6.73 (m, 4H, aromatic), 5.27 (s, 1H, CH, methine), 3.46 (m, 3H, CH₂, methylene), 3.22 (m, 6H, CH₂, methylene), 3.01 (m, 4H, CH₂, methylene), 2.30 (t, 2H, J=7.2 Hz, CH, methylene), 1.94 (t, 1H, J=6.6 Hz, CH, methine), 1.60 (m, 6H, CH₂, methylene), 1.38 (m, 4H, CH₂, methylene).

MS: HRMS (M+1): calculated 746.3435, found 746.4109.

### Synthesis of CY5.5-DSG

Compound 16, 15-DSG (1 mg, 0.0025 mmol] dissolved in 0.3 mL of 0.1 mol/L sodium borate (Na₂B₄O₇) buffer (pH-8.4) was mixed with Cy5.5 mono NHS ester (3.8 mg, 0.0033 mmol) in H₂O (0.1 mL) in the dark at 4° C. and stirred overnight. The purification of the crude product was carried out on a semipreparative HPLC system (1200 series pump, Agilent, Germany). Purification was performed on a Vydac, protein and peptide C₁₈ column (5 μm, 250×10 mm) The flow was 4 mL/min, with the mobile phase starting from 95% solvent A (0.1% trifluoroacetic acid in water) and 5% solvent B (0.1% trifluoroacetic acid in acetonitrile; 0 to 3 min) to 20% solvent A and 80% solvent B for 25 min The peak containing the desired compound at 8.8 min was collected, evaporated, and stored in the dark at −20° C. until use. The pure compound Cy5.5-DSG, 2.0 mg was obtained in 57% yield. The quality control analysis was performed with the same gradient system with a Vydac, protein and peptide C₁₈ column (5 μm, 150×4.6 mm) and flow was 1 mL/min, temperature 31° C.

MS: HRMS (M+K): calculated 1455.8290, found 1455.8415.

### Example 3

### Synthesis of Nutlin-2 Molecule

Nutlin-2, a family of cis-imidazoline analog, is a small molecule-MDM2 antagonist, based on the structural relationship between p53 and MDM2 and has the potential for target specificity. This molecule inhibited the interaction of MDM2-protein with a p53-like peptide with a potency that was 100-fold greater then a p53-derived peptide. Although not available commercially; Nutlin-2 was synthesized according to the reported procedure with modification for higher yield.

### Synthesis of Compound I

2-hydroxy-4-anisaldehyde (2.0 gm, 11.10 mmol) was dissolved in 30% ammonium hydroxide (30 mL) and 10 mL of acetonitrile (3:1), which resulted in the formation of a turbid solution. To this turbid solution, 2-iodoxybenzoic acid (6.22 gm, 22.20 mmol) was added slowly with constant stirring at 0° C. for 8 hr. The yellowish-brown solution becomes colorless which indicates completion of the reaction (TLC). The reaction mixture was filtered and evaporated under vacuum, and the residue was dissolved in dichloromethane (100 mL). The solution was washed with water (3×100 mL). The organic phase was dried (MgSO₄), evaporated to dryness, and purified on a silica gel column using 2% methanol in dichloromethane. The pure compound I (1.6 gm) was obtained in 81% yield.

MS: (M+1) calculated 178.19, found 178.43,

### Synthesis of Compound II

Compound I (1.0 gm, 5.64 mmol) was dissolved in anhydrous ethanol (5 mL, 84.6 mmol) in a dry flask. Acetyl chloride (3.2 mL, 45.15 mmol) was added slowly with stirring at 0° C. for 1 hr. The reaction flask was stoppered tightly and the stirring was continued at 25° C. for 48 hr, when TLC showed that no starting material remained. The reaction mixture was cooled to 0° C. and mixed slowly with saturated aqueous sodium bicarbonate solution, until gas evolution had ceased. The product was extracted into diethyl ether (50 mL) and the organic solution was washed with water (3×50mL) and brine and concentrated under reduced pressure to obtain the crude imidate. The crude residue was purified on a silica gel column using 5% methanol in dichloromethane. The pure compound II (1.0 gm) was obtained in 79% yield.

MS: (M+1) calculated 224.26, found 224.51.

### Synthesis of Compound III

4-bromobenzaldehyde (2.0 gm, 10.87 mmol) and ammonium acetate (3.33 gm, 43,24 mmol) was dissolved in water (5 mL) and heated at 120° C. for 5 hr when TLC showed that no starting material remained. The reaction mixture was cooled, and the precipitate was filtered off and washed with water and an 8% sodium hydroxide solution. The crude residue was recrystallized from 95% ethanol in water to obtain 4.6 gm of compound III (59% yield).

MS: (M+1) calculated 721.08, found 721.19.

### Synthesis of Compound IV

Compound III (4 gm, 5.56 mmol) was dissolved in concentrated sulfuric acid (10 mL) and water (30 mL) 1:3 ratio. The reaction mixture was boiled with stirring for 24 hr, after which TLC revealed that no significant starting material remained. The reaction mixture was cooled, and then the precipitate was filtered off and washed with water and an 8% sodium hydroxide solution. The crude residue was recrystallized from 90% ethanol in water to obtain 1.6 gm of compound IV (52% yield).

MS: (M+1) calculated 554.16, found 554.44.

### Synthesis of Compound V

Compound IV (1.0 gm, 1.81 mmol) was again strongly alkalized with saturated sodium hydroxide (5 mL) and stirred at room temperature for 2 hr. The reaction mixture was evaporated and the residue was purified on a silica gel column eluting with 3% isopropanol in ethyl acetate to produce a mixture of epimers. The mixture of epimers was separated by semipreparative HPLC. Separation was performed on an Econosil® C₁₈ column (10 μm, 10×250 mm). The flow was 4 mL/min, with the mobile phase starting from 90% solvent A (water) and 10% solvent B (acetonitrile) to 30% solvent A and 70% solvent B for 25 min to produce Va (0.28 gm, 42% yield) and Vb (0.2 gm, 30% yield) as solid powder.

MS: (M+1) calculated 371.29, found 371.61.

### Synthesis of Compound VI

Compound II (0.9 gm, 4,03 mmol) was dissolved in anhydrous ethanol (20 mL) in a dry flask under argon and triethylamine (2.8 mL, 20.15 mmol) was added followed by the addition of 4-dimethylaminopyridine (0.15 gm, 1.2 mmol). N-(4-bromobenzoyl)-meso-1, 2-di(4-bromophenyl)-1,2-diaminoethane, Va (1.6 gm, 4.43 mmol) was added and the reaction mixture is heated at reflux (95° C.) for 40 hr, after which, TLC revealed that no starting material remained. Aqueous sodium bicarbonate was added and solvent was evaporated under vacuum. The residue was dissolved in dichloromethane (50 mL) and washed with water (3×50 mL). The organic phase was dried (MgSO₄), evaporated to dryness and the crude product was purified on a silica gel column and using 2% methanol in dichloromethane as eluent. This afforded product VI as yellowish color powder (1.6 gm, 75% yield).

MS: (M+1) calculated 531.26, found 531.54.

### Synthesis of Compound VII

Compound VI (1.0 gm, 1.89 mmol) was dissolved in anhydrous methylene chloride (20 mL) in a dry flask under argon and triethylamine (1.3 mL, 9.4 mmol) was added followed by the addition of 4-dimethylaminopyridine (69 mg, 0.56 mmol). The reaction mixture was cooled to 0° C. and then phosgene (0.7 gm, 7.52 mmol, 20% in toluene) was added drop wise. The reaction mixture was warmed to room temperature and stirred for 1 hr, when TLC showed that no starting material remained. The solvent was evaporated under vacuum and crude product was directly purified on a silica gel column and using 2% methanol in dichloromethane as eluent. This afforded product VII as yellowish color powder (0.9 gm, 80% yield).

MS: (M+1) calculated 593.70, found 593.88.

### Synthesis of Compound VIII

2-piperazin-1-yl-ethanol (4.0 gm, 30.42 mmol) was dissolved in anhydrous methylene chloride (50 mL) at 0° C. under argon. Compound VII (1.0 gm, 1.69 mmol) was dissolved in anhydrous methylene chloride (20 mL), and then added dropwise to the above solution over 15 min. The reaction mixture was stirred at 0° C. for 2 hr and warmed to room temperature for 30 min, when TLC showed that no starting material remained. The reaction was taken up with 0.3 mL of water and evaporated under vacuum. The mixture was extracted with methylene chloride (100 mL) and washed with water (3×100 mL) and brine. The organic extracted was dried and evaporated. The crude product was purified on a silica gel column and using 5% methanol in dichloromethane as eluent to isolate VIII (0.8 gm, 69% yield).

The quality control analysis of the product was carried out on an analytical HPLC system. Analysis was performed on an Econosil® C₁₈ column (10 μm, 4.6×250 mm). The flow was 1 mL/min, with the mobile phase starting from 80% solvent A (water) and 20% solvent B (acetonitrile) to 20% solvent A and 80% solvent B for 25 min.

¹H NMR VIII (CDCl₃) δ: 7.55(d, 1H, J=8.4 Hz, CH, benzylidenimin), 7.29, 7.27,7.22, 7.18 (4s, 4H, aromatic), 6.85(dd, 4, J=8.4 Hz, aromatic), 6.56 (d, 1H, J=8.7 Hz, CH, benzylidenimin), 6.51(s, 1H, CH, benzylidenimin), 5.61 (d, 1H, J=9.6 Hz, CH, methine), 5.43 (d, 1H, J=9.6 Hz, CH, methine), 4.28-3.91(m, 2H, CH₂, methylene), 3.88 (s, 3H, CH₃), 3.59 (t, 2H, J=5.4 Hz, CH₂, methylene), 3.18 (bs, 4H, CH₂, methylene), 2.44 (t, 2H, J=5.1 Hz, CH₂, methylene), 2.16 (bs, 4H, CH₂, methylene), 1.45 (t, 3H, J=6.9 Hz, CH₃).

High resolution MS: M+1, calculated 687.4341, found 687.3608.

### Example 4

### Synthesis of Bifunctional Molecules

### HSP Binding Portion

15-DSG (FIG. 1) binds Hsp70 (K_{D}=4 μM) and stimulates its steady state ATPase activity. This molecule has been chosen as the Hsp70 binding moiety of a bifunctional molecule.

### Inhibitor of P53-MDM2 Interaction Portion

Nutlin-2 is a small-molecule inhibitors, it can bind MDM2 in the p53 pocket and activate p53 pathway in cancer cells, leading to cell cycle arrest, apoptosis, and growth inhibition of human tumor xenografts in nude mice. Nutlin-2 was chosen for the bifunctional molecule for its good binding affinity (IC₅₀=0.14 μM) with MDM2.

### Linker

To prepare these novel bifunctional molecules, suitable linkers were selected to efficiently link the Hsp70 binding portion and the p53-MDM2 binding moieties. Selection of functional groups to permit labeling of the bifunctional molecules with radionuclides such as ¹⁸F to carry out PET imaging study was also desirable. Four amino acids with an amino group and two carboxyl groups in different length of carbon chains were chosen as linkers. These were all commercially available.

### Synthesis of Compound B1

Compound VIII (1.0 gm, 1.46 mmol) was dissolved in anhydrous methylene chloride (20 mL) in a dry flask under argon and N,N′-dicyclohexylcarbodiimide (0.9 gm, 4.38 mmol) was added followed by the addition of 4-dimethylamino pyridine (0.19 gm, 1.46 mmol). Linker (b), Fmoc-Aad (OtBu)-OH (0.7 gm, 2.04 mmol) was dissolved in anhydrous methylene chloride (5 mL) and then added dropwise to the above mixture for over 10 min. The reaction mixture was stirred at temperature for 4 hr, after which TLC revealed no starting material remained. The N,N′-dicyclohexylcarbodiimide salt is separated from the reaction mixture by filtration. The filtrate was concentrated and purified by silica gel column using 5% methanol in dichloromethane as eluent to isolate B1 (1.1 gm; 69% yield).

MS: (M+1) calculated 1108.91, found 1109.21.

### Synthesis of Compound B2

Compound B1 (0.9 gm, 0.82 mmol) was dissolved in anhydrous methylene chloride (10 mL) in a dry flask under argon at 0° C. Anhydrous trifluoroacetic acid (3.2 mL, 41.13 mmol) was added dropwise to the above mixture for over 20 min The reaction mixture was allowed to stir at 0° C. for 5 hr, after which TLC revealed that no starting material remained. The mixture was extracted with methylene chloride (30 mL) and washed with water (3×30 mL) and brine. The organic layers were dried and evaporated. The crude product was purified on a silica gel column and using 3% methanol in dichloromethane as eluent to isolate B2 (0.7 gm) in 80% yield,

MS: (M+1) calculated 1052.84, found 1052.98.

### Sunthesis of Compound B3

Compound B2 (0.6 gm, 0.58 mmol) was dissolved in anhydrous acetonitrile (10 mL) and tetrahydrofuran (10 mL) (1:1) in a dry flask under argon and N-hydroxysuccinimide (86 mg, 0.76 mmol) was added followed by the addition of N,N′-dicyclohexylcarbodiimide (0.16 gm, 0.76 mmol). The reaction mixture was stirred at room temperature for 30 hr, after which TLC revealed that no starting material remained. The solvent was evaporated under vacuum. The residue was dissolved in dichloromethane (30 mL) and the solution washed with water (3×30 mL). The organic phase was dried (MgSO₄), evaporated to dryness and the crude product was purified on a silica gel column and using 2% methanol in dichloromethane as eluent. This afforded product B3 as white powder (0.44 gm, 67% yield).

MS: (M+1) calculated 1149.71, found 1149.95.

### Synthesis of Compound B4

Compound 16, 15-DSG (0.1 gm, 0.26 mmol) and triethylamine (0,11 mL, 0.78 mmol) were dissolved in N,N-dimethylformamide (5 mL) and sonicated for 10 min under argon. Compound B3 (0.58 gm, 0.52 mmol) was dissolved in N,N-dimethylformamide (3 mL) and added to the above solution at 0° C., warmed to room temperature and stirred overnight until TLC showed that no starting material remained. The solvent was evaporated and the crude product was purified on a semipreparative HPLC system. Purification was performed on an Econosil® C₁₈ column (10 μm, 250×10 mm) The flow rate was 4 mL/min, with the mobile phase starting from 90% solvent A (0.1% trifluoroacetic acid in water) and 10% solvent B (0.1% trifluoroacetic acid in methanol) to 10% solvent A and 90% solvent B at 20 min The pure compound B4 (0.2 gm, 55% yield) was collected at 18.36 min, dried, evaporated and stored in at −20° C. until it was deprotected (F-moc group).

High-resolution MS: M+1, calculated 1422.3188, found 1422.3204.

### Synthesis of Compound B5

Compound B4 (0.2 gm, 0.14 mmol) was dissolved in dichloromethane (5 mL) under argon. This solution was treated with piperidine (0.11 mL, 1.12 mmol) and stirred at room temperature for 3 hr, after which TLC revealed that no starting material remained. The solvent was evaporated and the crude product was purified on a semipreparative HPLC system. Purification was performed on an Econosil® C₁₈ column (10 μm, 250×10 mm). The flow rate was 4 mL/min, with the mobile phase starting from 80% solvent A (0.1% trifluoroacetic acid in water) and 20% solvent B (0.1% trifluoroacetic acid in methanol) to 20% solvent A and 80% solvent B for 20 min The pure compound B5 (0.14 gm, 84%) was collected, dried, evaporated and stored at −20° C.

¹H NMR B5 (CD₃OD) δ: 7.72(d, 1H, J=9.0 Hz, CH, benzylidenimin), 7.42 (d, 2H, J=8.5 Hz, aromatic), 7.42 (d, 2H, J=8.0 Hz, aromatic), 7.07(d, 2H, J=8.5 Hz, aromatic), 6.99(d, 2H, J=8.0 Hz, aromatic), 6.86(m, 4H, amine and amide), 6.20 (d, 1H, J=11.0 Hz, CH, benzylidenimin), 6.00(d, 111, J=10.5 Hz, CH, benzylidenimin), 4.46 (m, 2H, CH, methine), 4.29 (m, 2H, CH₂, methylene), 4.14(t, 2H, J=6.0 Hz, CH, methine), 3.98 (s, 3H, CH₃), 3.47 (bs, 3H, CH₂, methylene), 3.40 (s, 3H, CH₂, methylene), 3.31 (m, 4H, CH₂, methylene), 3.19 (m, 4H, CH₂, methylene), 3.03 (m, 5H, CH₂, methylene), 2.82 (m, 4H, CH₂, methylene), 2.49 (m, 2H, CH₂, methylene), 2.32 (t, 2H, J=7.5 Hz, CH₂, methylene), 2.19 (m, 3H, CH₂, methylene), 1.89 (m, 2H, CH₂, methylene), 1.69 (m, 8H, CH₂, methylene), 1.48 (t, 3H, J—7.0 Hz, CH₃), 1.42 (m, 4H, CH₂, methylene).

High-resolution MS: M+1, calculated 1200.0801, found 1200.0854.

### Example 5

### Synthesis of Fluoronated Bifunctional Molecules

### Synthesis of Compound 19

Compound B5, bifunctional molecule (30 mg, 0.03 mmol) and triethylamine (0.11 μL, 0.07 mmol) were dissolved in dimethylsulfoxide (1 mL) under argon. Compound 17 (9 mg, 0.04 mmol) was added to the above solution and heated at 90° C. for 25 min. The reaction mixture was then cooled to room temperature and the solvent evaporated under high vacuum. The crude residue was purified on a semipreparative HPLC system on an Econosil® C₁₈ column (10 μm, 250×10 mm) with a flow rate of 4 mL/min using a gradient system from which the product 19 was isolated.

### Sunthesis of FAM-Labeled Bifunctional Molecules

The synthesis of another fluorescent-bifunctional molecule was carried out as follows: Bifunctional molecule (1.2 mg, 1 μmol and triethylamine were added to 6-carboxyfluorescein (FAM) succinimidyl ester (2.4 mg, 5 mop in DMSO (1 mL) in the dark at ambient temperature. After stirring overnight, the reaction was quenched by adding 200 μL of 5% acetic acid (HOAc). The purification of the crude product was carried out on a semipreparative reversed-phase HPLC system. The peak containing the FAM-bifunctional molecule conjugate (BF-FAM) was collected, lyophilized and stored in the dark at −20° C. until use.

### Example 6

### In Vitro Analyses of Bifunctional Molecules

### Tissue Culture Studies

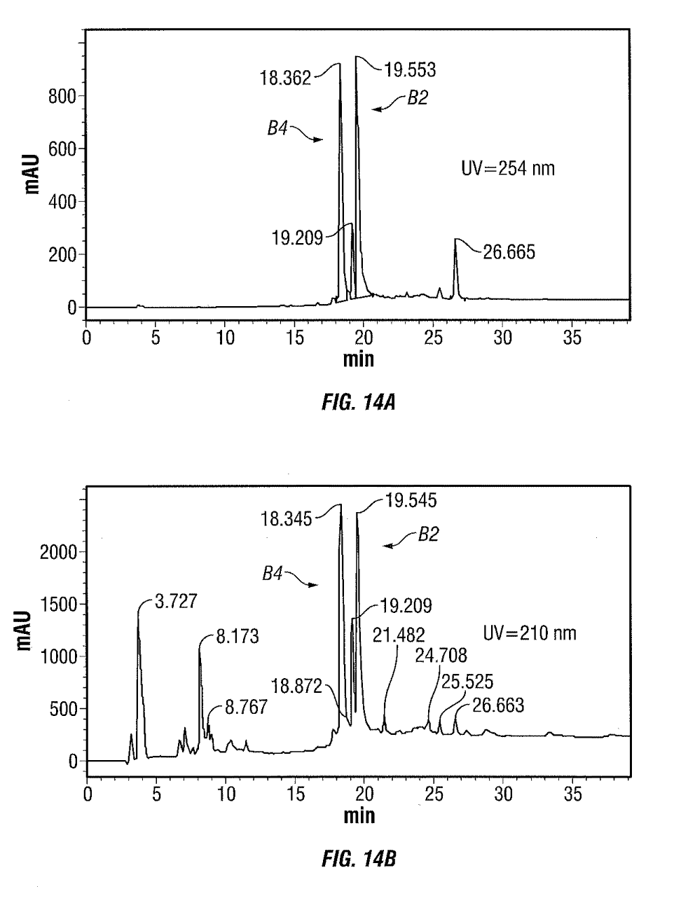

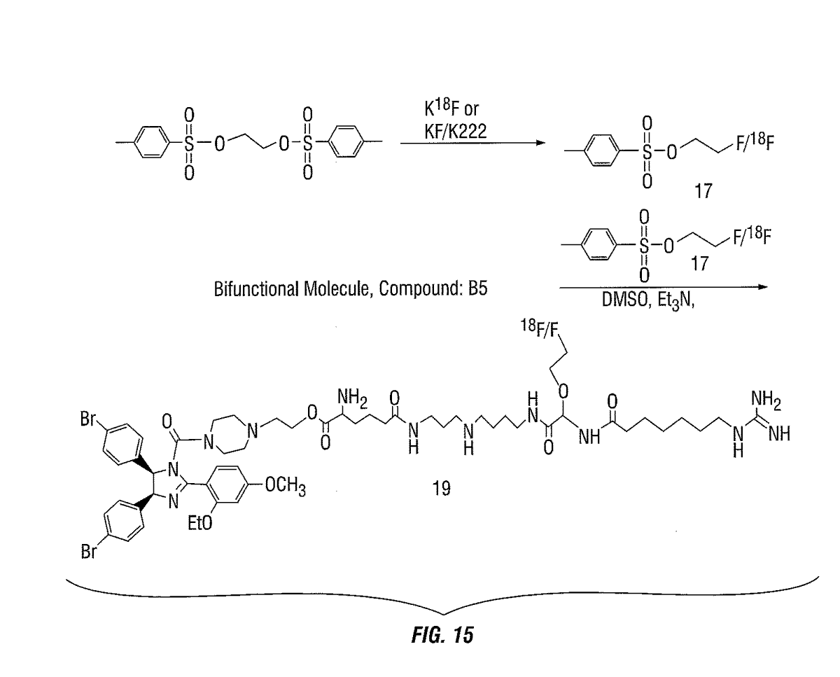

For immunohistochemical staining, A549 cells were plated at a density of approximately 10,000 cells per chamber in a 96-well clear bottom black plate and grown overnight at 37° C. Fixing was achieved with 4% formaldehyde and the subsequent staining procedure was performed as described in the antibody supplier's instructions (R & D Systems, Inc., Minneapolis, Minn., USA). As an additional positive control, A549 cells were treated with CuSO₄ for 24 hours as previously described (Neuhaus-Steinmetz and Rensing, 1997; and Wiegant et al., 1999), followed by the staining procedure using a monoclonal anti-HSP70 antibody (catalog number MAB1663) followed by fluorescein-labeled secondary antibody detection (FIG. 14A, FIG. 14B, FIG. 14C, and FIG. 14D). Nuclear counterstaining with DAPI (4′,6-diamidino-2-phenylindole) was also performed. For compound uptake studies, cells were grown either at 37° C. (control) or at 45° C. for 10 min by transferring the cell plate into an incubator, followed by a period of recovery at 37° C. Purified Cy5.5-labeled DSG was then directly added to the culture medium at an approximate concentration of 1 μM and incubated for one hour. Cells were then washed with warm PBS twice and then visualized on a fluorescence microscope equipped with a Cy5 filter (excitation 620 nm, emission 700 nm) at an optical magnification of 20× (FIG. 15). Phase-contrast images were obtained with each view.

To further assess the results of the synthesized compounds in vitro, and to test the hypothesis that bifunctional molecules with MDM2 and HSP70 specificities could bind to their cognate protein targets, the approach of Vassilev et al, (2004) was used to determine the cytotoxic effect of the synthesized bifunctional compounds in accordance with one aspect of the present invention. Approximately 10³ MDA-MB-435S (human melanoma) with known p53 mutation^{76, 77} and HCT 116 (human colorectal carcinoma) with wild type p53⁷⁸ were plated on 96-well polystyrene plates in Leibovitz's L-15 or McCoy's 5a medium, respectively and allowed to grow exponentially at 37° C.

For hyperthermia treatment, cells were placed in a tissue culture incubator preset to 46.5° C. After accounting for a ramp-up period, cells were maintained at this temperature for 5 min. Under these conditions, no heat-induced cytotoxicity was observed in the cell lines tested. Immediately after heating, the original media was exchanged with pre-warmed (37° C.) medium containing no drug (negative control) or one of various concentrations of the bifunctional molecule (e.g., 0.5, 1.0, 2.0, 5.0 or 10 μM), cells were then returned to a tissue culture incubator set at 37° C. and grown for another 5 days.

Cell viability was determined with the CellTiter 96® Aqueous Assay (Promega, Madison, Wis.) which is based on MTS (tetrazolium compound (3-(4, 5 dimethylthiazol-2-yl)-5-(3-carboxymethoxyphenyl)-2-(4-sulfophenyl)-2H-tetrazolium, inner salt) and the electron coupling reagent, phenazine methosulfate and optically measured at 492 nm in a standard plate reader (BMG Labtech, Durham, N.C.) following the manufacturer's protocols.

As predicted from the known p53 mutation, no significant cytotoxicity was observed with MDS-MD-435S cells at any concentration tested regardless of hyperthermia treatment. However, viability was reduced in HCT 116 cells by approximately 50% at the 10 μM dose; this cytotoxic effect was slightly more pronounced at the 2, 5, and 10 μM concentrations following heat treatment. This data paralleled the Western blotting results using an anti-hsp70 monoclonal antibody (R&D Systems, Minneapolis, Minn.).

Interestingly, for MDA-MB-435S cells, the levels of HSP70 protein were marginally detectable when unheated (baseline) but was significantly overexpressed upon heat-induction. In contrast, HCT 116 cells demonstrated a moderately high basal level of HSP70 protein, which was further increased upon heat-induction. Collectively, these data suggested that the bifunctional molecule exerts its effect on HCT 116 cells (wild-type p53) but not on MDA-MB-435S cells (mutant p53), and was facilitated by heat-treatment, consistent with a dual mechanism of action that targets HSP70 via the 15-DSG moiety and disruption of the MDM2-p53 interaction by the Nutlin moiety.

### Example 7

### In Vivo Analyses of Bifunctional Molecules

### In Vivo Analysis of HSP Induction

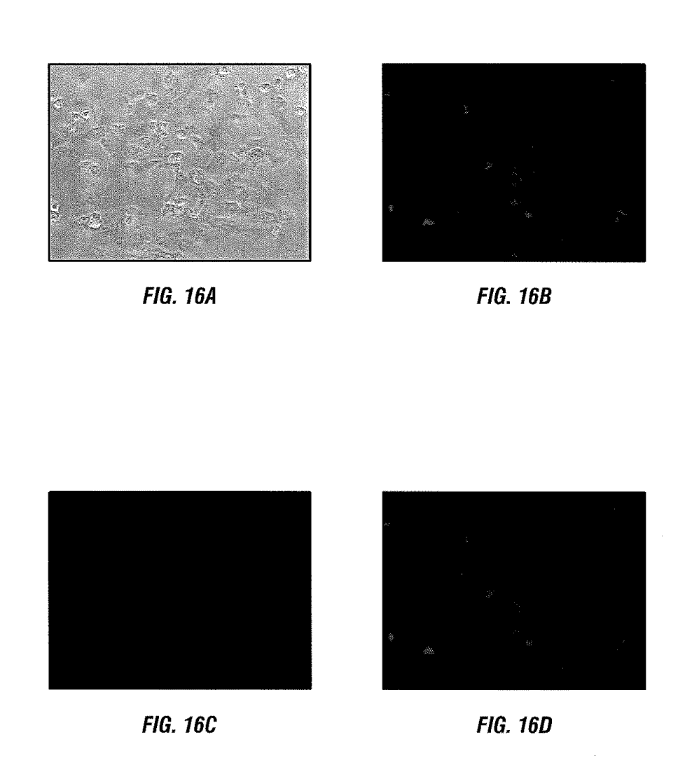

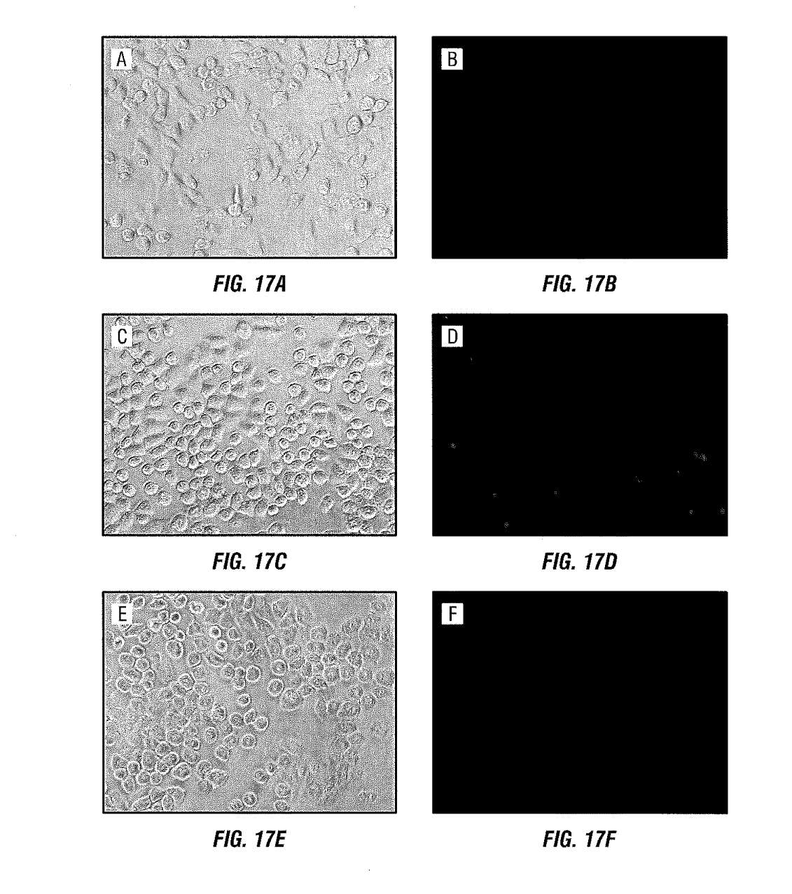

Six-week-old BALB/c nu/nu female mice were purchased from Charles River Laboratories and fed a regular diet. Mice were utilized in a heat-induced Hsp70 imaging study. A heating apparatus was constructed to permit localized deposition of heated water to one (right hind) limb of each mouse (FIG. 16 and FIG. 17). Mice were induced with 4% isoflurane and with continued anesthesia on 2% throughout the entire heating procedure. Each mouse was positioned into the heating apparatus and monitored during the entire heating procedure. Heating times were varied from 5 min to 20 min at 45° C. Additional tested temperatures were 43° C. and 44° C. for 10 min (data not shown). After 10 min of heating, animals were removed from the apparatus and allowed to recover for 30 min at ambient temperature. During the 10-min heating period, no adverse effects were observed. However, following the initial heating period, localized edema and erythema were noted in the heated limb (data not shown). Upon completion of heating, mice were removed from the apparatus and allowed to recover at ambient temperature for 30 min, 3 hr, or 5 hr.

After the recovery period, approximately 0.1 pg of the DSG-Cy5.5 was intravenously administered in a total volume of 50 μL via the tail vein and imaged in a Xenogen/Caliper MS-200 bioluminescence/fluorescence system using the appropriate Cy5.5 filters with 0.15 sec exposure time and medium binning.

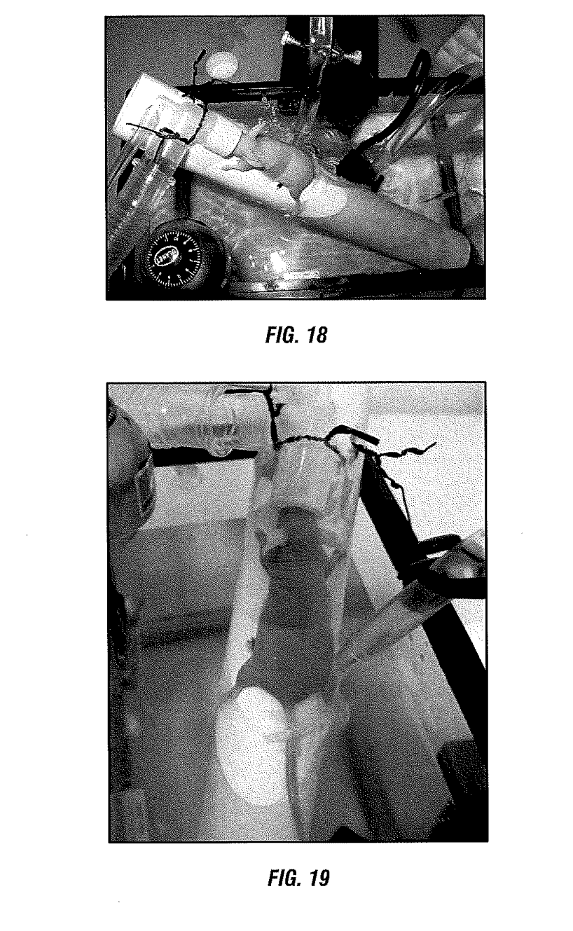

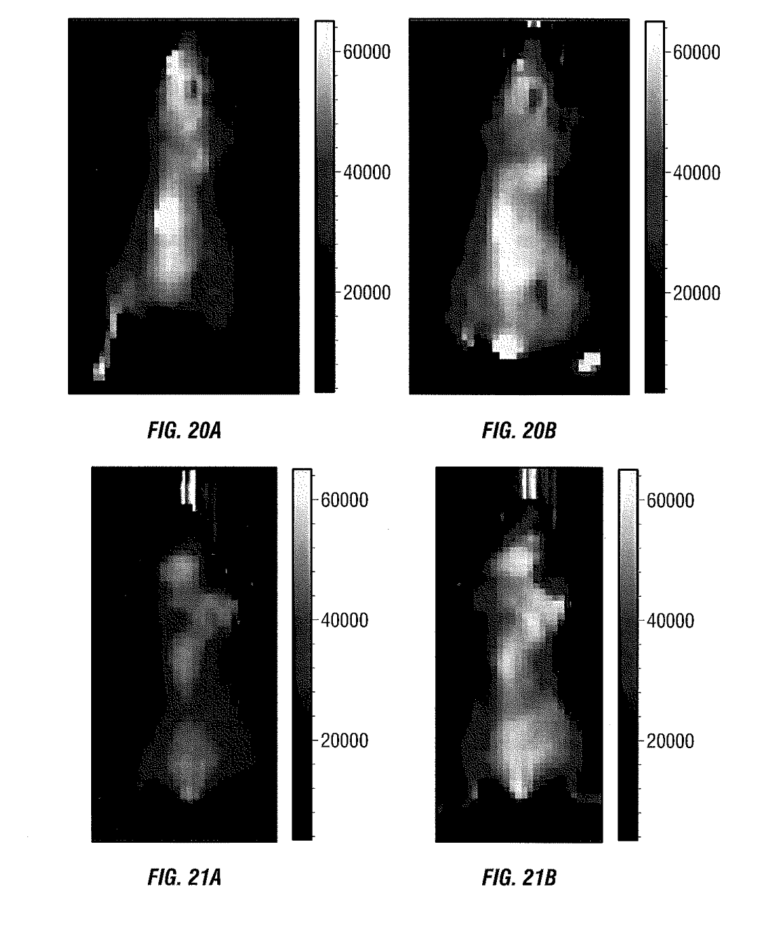

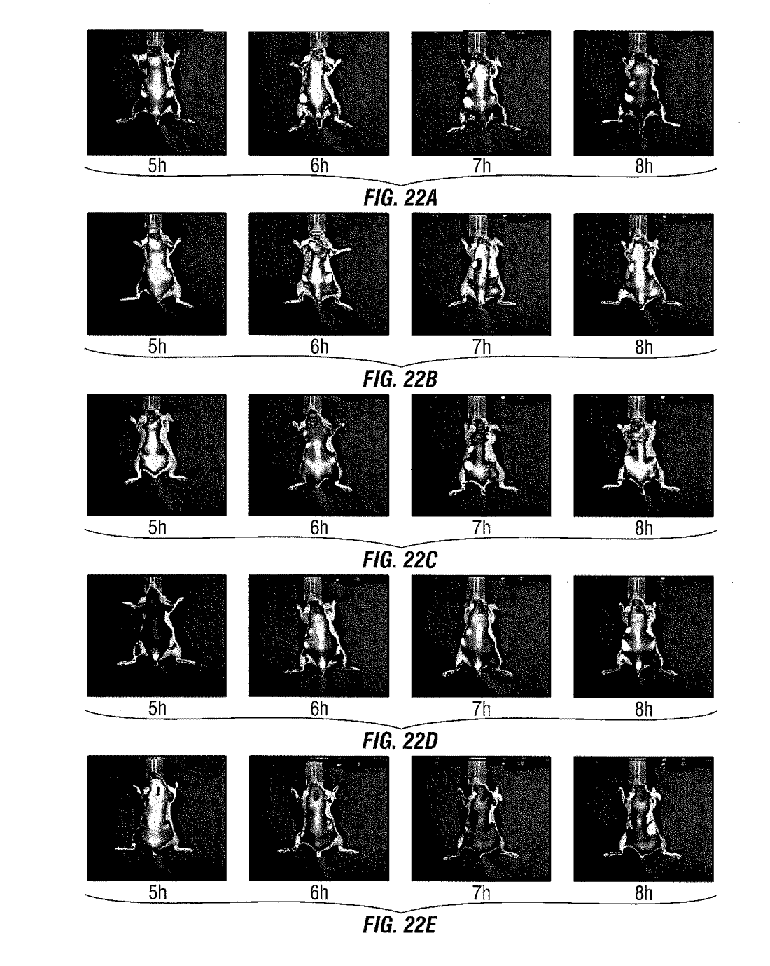

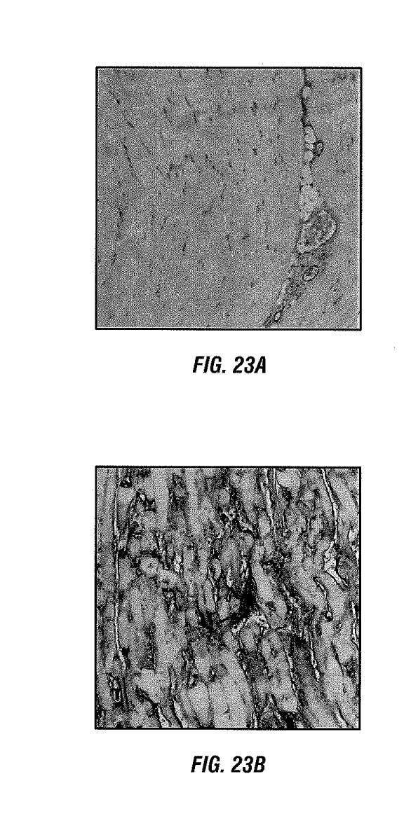

During image acquisitions, mice were continuously anesthetized with 2% isoflurane and warmed in the imaging chamber. Results from a study testing 45° C. for 6 to 10 min are shown in FIG. 22A, FIG. 22B, FIG. 22C, FIG. 22D, and FIG. 22E. As evident from these images, increased signal intensity in the right hind limb was detectable under certain conditions (indicated by arrows). Following a period of approximately 24 hrs, a second intravenous dose of compound was administered and imaged immediately and at 1 hr post-injection, demonstrating persistent signal detectable in the treated limb at 24 hrs' post heating (data not shown). Tissue harvested at 6 hours' post-heating were stained for HSP70 and compared to the contralateral untreated limb (FIG. 2A and FIG. 23B). Intense staining was observed in the heated tissue, providing strong evidence for the proposed mechanism of action of the DSG-Cy5.5 targeting in the experimental system (FIG. 18 and FIG. 19). Image processing was performed with the Living Image) version 3.0 software.

### In Vivo Analysis of a FAM-Labeled Bifunctional Molecule

In another study demonstrating the effectiveness of the targeting compounds in accordance with one aspect of the invention, experimental animals were treated with heated water to the right hind limb. Following a recovery period of 4 hours, 20 nmol of the bifunctional molecule conjugated to fluorescein (BF-FAM) was intravenously administered without any observable immediate or delayed adverse effects. As a control, 6-carboxyfluorescein (6-FAM) was administered at the same dose (20 nmol) in the same manner. Due to the high intrinsic autofluorescence of the dermis, experimental mice were sacrificed, the dermis dissected, and imaged on a small animal dedicated optical system (Xenogen/Caliper IVIS-200) using the appropriate excitation/emission filter set.

In contrast to the negative control, following approximately 4 hours' post-injection of BF-FAM (corresponding to 8 hrs post hyperthermia treatment), a significant level of fluorescence signal was observed in the heated limb tissue. A minimal level of fluorescence was observed in the heated limb with the 6-FAM control, likely reflecting transiently increased tissue edema due to the hyperthermia treatment. However, under these heating conditions no obvious tissue injury was observed to suggest vascular compromise or cellular necrosis.

It was determined that under these hyperthermia treatment parameters, the levels of HSP70 expression were robustly increased. Internal organs harvested at the time of sacrifice provided a gross estimate of biodistribution of systemically administered BF-FAM relative to the 6-FAM control. Significant fluorescence was observed in the gastrointestinal tract. However, relative to control there was slightly more fluorescence signal the liver in the BF-FAM subject, suggesting a hepatic mode of metabolism of the bifunctional molecule.

These examples demonstrated the novel approaches described herein for imaging an inducible target using an endogenous protein involved in stress response. In these studies, brief heat treatment to a desired tissue site was sufficient to induce HSP70 as demonstrated by immunohistochemical analysis of treated tissue. This effect was observed both in tissue culture as well as in experimental mice with comparable findings.

### Example 8

### Synthesis of Bifunctional Molecule Geldanamycin-DOX

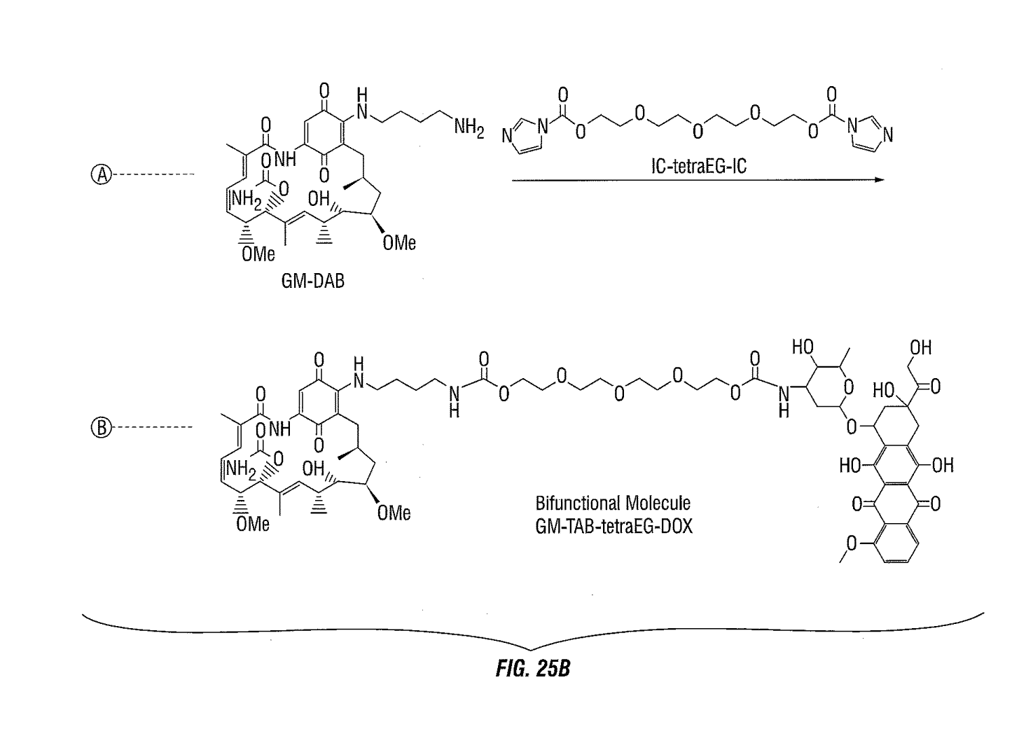

Geldanamycin is an Hsp90 inhibitor, and one of its derivatives, 17-AAG, is currently in phase III clinic trials for cancer therapy. A bifunctional molecule has been developed in which geldanamycin is linked to doxorubicin using a polyethylene glycol (PEG) linker. The synthesis of this bifunctional molecule is shown in FIG. 25.

GM-BDA: 30 mg GM (50 μmol) was dissolved in 0.5 ml DMF cooled to 0° C., 0.2 ml BDA (1 mmol/ml, 200 μmol) was added and the mixture was stirred for 10 min. The purification of the crude product was carried out on a semi-preparative reversed-phase HPLC system (Agilent 1200 series). The mass spectrum of GM-BDA: m/z 617.1 (100, [M+H]⁺, calculated 616.3); 1233.1 ([2M+H]⁺).

GM-BDA-tetraEG-IC: To 3 μmol GM-BDA in 1 mL DMSO, 26 mg IC-tetraEG-IC (68 μmol) was added. The mixture was heated at 120° C. for 10 min in a microwave synthesizer. The purification of the crude product was carried out on a semi-preparative reversed-phase HPLC system (Agilent 1200 series). The mass spectrum of GM-BDA-tetraEG-IC: m/z 931.1 (100, [M+H]⁺, calculated 930.5).

GM-BAD-tetraEG-DOX: To 3 μmol GM-BDA-tetraEG-IC in 1 mL DMSO, 5 mg doxorubicin hydrochloride was added. The mixture was cooled to 0° C. and stirred for 12 hr. The purification of the crude product was carried out on a semi-preparative reversed-phase HPLC system (Agilent 1200 series). The mass spectrum of GM-BDA-tetraEG-DOX: m/z 1406.1 (100, [M+H]⁺, calculated 1405.6).

### REFERENCES

The following references, to the extent that they provide exemplary procedural or other details supplementary to those set forth herein, are specifically incorporated herein in their entirety by express reference thereto:

U.S. Pat. No. 7,393,478, entitled “Therapy for human cancers using cisplatin and other drugs or genes encapsulated into liposomes.”

U.S. Pat. No. 7,384,923, entitled “Liposomes.”

U.S. Pat. No. 7,371,404, entitled “Amphoteric liposomes and their use.”

U.S. Pat. No. 7,354,567, entitled “Method of encapsulating metal complex within liposomes.”

U.S. Pat. No. 7,312,206, entitled “Sterol derivatives, liposomes comprising sterol derivatives and method for loading liposomes with active substances.”

U.S. Pat. No. 7,273,620, entitled “Triggered release of liposomal drugs following mixing of cationic and anionic liposomes.”

U.S. Pat. No. 7,262,173, entitled “Chemosensitizing with liposomes containing oligonucleotides.”

U.S. Pat. No. 7,205,273, entitled “Fusogenic liposomes.”

U.S. Pat. No. 7,153,933, entitled “Solid phase method for synthesis peptide-spacer-lipid conjugates, conjugates synthesized thereby and targeted liposomes containing the same.”

U.S. Pat. No. 7,153,490, entitled “Liposomes encapsulating anticancer drugs and use thereof in the treatment of malignant tumors.”

U.S. Pat. No. 7,150,883, entitled “Self forming, thermodynamically stable liposomes and their applications.”

U.S. Pat. No. 7,067,697, entitled “Cationic liposomes for gene transfer.”

U.S. Pat. No. 6,989,153, entitled “Radiation sensitive liposomes.”

U.S. Pat. No. 6,964,778, entitled “Temperature controlled content release from liposomes.”

U.S. Pat. 6,958,160, entitled “Self forming, thermodynamically stable liposomes and their applications.”

U.S. Pat. No. 6,767,554, entitled “Use of complexes among cationic liposomes and polydeoxyribonucleotides and medicaments.”

U.S. Pat. No. 6,743,638, entitled “Detection system using liposomes and signal modification.”

U.S. Pat. No. 6,726,926, entitled “Gene-entrapped liposomes preparation and process for the preparation thereof.”

U.S. Pat. No. 6,610,322, entitled “Self forming, thermodynamically stable liposomes and their applications.”

U.S. Pat. No. 6,610,304, entitled “Liposomes containing multiple branch peptide constructions for use against human immunodeficiency virus.”

U.S. Pat. No. 6,596,543, entitled “Use of liposomes of defined composition and size for the preparation of prothrombin time reagents.”

U.S. Pat. No. 6,596,305, entitled “Method of controlling the size of liposomes.”

U.S. Pat. No. 6,593,294, entitled “Pharmaceutical composition comprising Factor VIII and neutral liposomes.”

U.S. Pat. No. 6,592,843, entitled “Radioactive therapeutic liposomes.”

U.S. Pat. No. 6,511,677, entitled “Polymerizable fatty acids, phospholipids and polymerized liposomes therefrom.”

U.S. Pat. No. 6,511,676, entitled “Therapy for human cancers using cisplatin and other drugs or genes encapsulated into liposomes.”

U.S. Pat. No. 6,469,084, entitled “Process for preparing an aqueous composition in gel form and compositions obtainable from this process, especially a composition containing vesicles, in particular liposomes.”

U.S. Pat. No. 6,458,381, entitled “Lipids and their use, for example, in liposomes.”

U.S. Pat. No. 6,451,338, entitled “Liposomes containing particulate materials.”

U.S. Pat. No. 6,426,086, entitled “pH-sensitive, serum-stable liposomes.”

U.S. Pat. No. 6,417,326, entitled “Fusogenic liposomes.”

U.S. Pat. No. 6,387,397, entitled “Polymerized liposomes targeted to M cells and useful for oral or mucosal drug delivery.”

U.S. Pat. No. 6,380,359, entitled “Liposomes comprising peptide antigens derived from X protein of hepatitis B virus.”

U.S. Pat. No. 5,540,936, entitled “Method of producing liposomes.”

PCT Intl. Pat. Appl. Publ. No. WO95/08348.

PCT Intl. Pat. Appl. Publ. No. WO 2003/53462.

PCT Intl. Pat. Appl. Publ. No. WO 2004/43407.

Altschul et al., J. Mol. Biol., 215:403-410, 1990.

Alysius M M et al., “Dendritic cell biology, dysfunction and immunotherapy in gastrointestinal cancers,” Surgeon, 4:195-210, 2006.

Bengel et al., “Cell-based therapies and imaging in cardiology,” Eur. J. Nucl. Med. Mol. Imaging, 32 Suppl 2:S404-16, 2005.

Benjamin and McMillian, Circ. Res., 83:117-132, 1998.

Bulte J W and Kraitchman D L, “Monitoring cell therapy using iron oxide MR contrast agents,” Curr. Pharm. Biotechnol., 5:567-584, 2004.

Calderwood et al., “Message in a bottle: role of the 70-kDa heat shock protein family in anti-tumor immunity,” Eur. J. Immunol., 35(9):2518-2527, 2005.

Chang et al., “Overview of stem cells and imaging modalities for cardiovascular diseases,” J. Nucl. Cardiol., 13:554-569, 2006.

Chen et al., “Synergistic activation of p53 by inhibition of MDM2 expression and DNA damage,” Proc. Natl. Acad. Sci. USA, 95(1):195-200, 1998.

Chene et al., “A small synthetic peptide, which inhibits the p53-hdm2 interaction, stimulates the p53 pathway in tumour cell lines,” J. Mol. Biol., 299(1):245-253, 2000.

Chene P., “Inhibiting the p53-MDM2 interaction: an important target for cancer therapy,” Nature Rev., 3(2):102-109, 2003.

Chene P., “Inhibition of the p53-MDM2 interaction: targeting a protein-protein interface,” Mol. Cancer Res., 2:20-28, 2004.

Ciocca D R, and Calderwood S K, “Heat shock proteins in cancer: diagnostic, prognostic, predictive, and treatment implications,” Cell Stress and Chaperones, 10(2):86-103, Summer 2005.

Conley et al., Vaccine 12:445-451, 1994.

Feng and Doolittle, J. Mol. Evol., 35:351-360, 1987.

Flanagan et al., “Tissue-specific HSP70 response in animals undergoing heat stress,” Am. J. Physiol., 268(1 Pt 2):R28-32, 1995.

Fotouhi N, and Graves B, “Small molecule inhibitors of p53/MDM2 interaction,”Curr. Top. Med. Chem., 5:159-165, 2005.

Freedman et al., “Functions of the MDM2 oncoprotein,” Cell. Mol. Life Sci., 55(1):96-107, 1999.

Fry, D C, and Vassilev L T, “Targeting protein-protein interactions for cancer therapy,” J. Mol. Med., 83:955-963, 2005.

Frydman et al., Nature, 370:111-117, 1994.

Garrido et al., “Heat shock proteins 27 and 70: anti-apoptotic proteins with tumorigenic properties,” Cell Cycle (Georgetown, Tex.), 5(22):2592-2601, 2006.

Gestwicki J E, Crabtree G R, and Graef I A, “Harnessing chaperones to generate small-molecule inhibitors of amyloid beta aggregation,” Science, 306:865-869, 2004.

Hahn et al., “Mammalian stress proteins HSP70 and HSP28 coinduced by nicotine and either ethanol or heat,” Molec. Cell. Biol., 11(12):6034-6040, 1991.

Hendrick and Hartl, Ann. Rev. Biochem., 62:349-384, 1993.

Henikoff and Henikoff, Proc. Natl. Acad. Sci. USA, 89:10915, 1989.

Higgins and Sharp, Comput. Appl. Biosci., 5:151-153, 1989.

Holcombe et al 2002, J. Immunol., 4982-4989.

Jaattela M., “Escaping cell death: survival proteins in cancer,” Exper. Cell Res., 248(1):30-43, 1999.

Karlin and Altschul, Proc. Natl. Acad. Sci, USA, 90:5873-5787, 1993.

Kohler and Milstein, Nature, 256:495-497, 1975.

Kregel et al., “HSP70 accumulation in tissues of heat-stressed rats is blunted with advancing age,” J. Appl. Physiol., 79(5):1673-1678, 1995.

Kregel K C, and Moseley P L., “Differential effects of exercise and heat stress on liver

HSP70 accumulation with aging,” J Appl Physiol., 80(2):547-551, 1996.

Kregel K C., “Heat shock proteins: modifying factors in physiological stress responses and acquired thermotolerance,” J Appl. Physiol., 92(5):2177-2186, 2002.

Kubbutat M H, Jones S N, Vousden K H. Regulation of p53 stability by Mdm2. Nature, 387(6630):299-303, 1997.

Landry et al., “Synthesis and degradation of heat shock proteins during development and decay of thermotolerance,” Cancer Res., 42(6):2457-2461, 1982.

Landry J, Samson S, Chretien P, Hyperthermia-induced cell death, thermotolerance, and heat shock proteins in normal, respiration-deficient, and glycolysis-deficient Chinese hamster cells. Cancer Res., 46(1):324-327, 1986.

Lane D P., “Exploiting the p53 pathway for cancer diagnosis and therapy,” Brit. J. Cancer, 80 Suppl 1:1-5, 1999.

Li G C, and Mak J Y., “Induction of heat shock protein synthesis in murine tumors during the development of thermotolerance,” Cancer Res., 45(8):3816-3824, 1985.

Lou, M., Nature, 443:37-38, 2006.

McCurry K R et al., “Regulatory dendritic cell therapy in organ transplantation. Transpl. Int., 19:525-53 8, 2006.

Momand et al., “The MDM2 gene amplification database. Nucl. Acids Res., 26(15):3453-3459, 1998.

Nadler et al, Science, 258:484, 1992.

Needleman and Wunsch, J. Mol. Biol., 48:443, 1970.

Neuhaus-Steinmetz, U. and L. Rensing, “Heat shock protein induction by certain chemical stressors is correlated with their cytotoxicity, lipophilicity and protein-denaturing capacity,” Toxicology, 123(3):185-95, 1997.

Neuhaus-Steinmetz, U. and L. Rensing, 1997, Toxicology, 123(3); 185-95,

Nylandsted et al., “Selective depletion of heat shock protein 70 (Hsp70) activates a tumor-specific death program that is independent of caspases and bypasses Bcl-2,” Proc. Natl. Acad. Sci. USA, 97(14):7871-7876, 2000.

Pearson and Lipman, Proc. Natl. Acad. Sci. USA, 85:2444, 1988.

Powers et al., FEBS Letters, 581, 3758-3769, 2007.

Ramya et al., Bioch. Biophys. Res. Com., 348:585-592, 2006.

Mitossa, F., “A new puffing pattern induced by heat shock and DNP in Drosophila,” Experimentia, 18:571-573, 1962.

Schaeider et al., Ann. N.Y. Acad. Sci., 973:8-12, 2002.

Schmitt et al., “Intracellular and extracellular functions of heat shock proteins: repercussions in cancer therapy,” J. Leukocyte Biol., 81(1):15-27, 2007.

Smith and Waterman, Adv. Appl. Math., 2:482, 1981.

Soti et al., “Heat shock proteins as emerging therapeutic targets,” Brit. J. Pharmacol., 146(6):769-780, 2005.

Stamm et al., “Stem cell therapy for ischemic heart disease: beginning or end of the road?” Cell Transplant., 15Suppl 1:547-56, 2006.

Subjeck J R, and Shyy T T., “Stress protein systems of mammalian cells,” Am. J. Physiol., 250(1 Pt 1): C1-17, 1986.

Tolman et al., Int. J. Pep. Prot. Res., 41:455-466, 1993.

Vassilev et al., “In vivo activation of the p53 pathway by small-molecule antagonists of MDM2,” Science, 303(5659):844-848, 2004.

Wiegant et al., “Stimulation of survival capacity in heat shocked cells by subsequent exposure to minute amounts of chemical stressors; role of similarity in hsp-inducing effects,” Hum. Exp. Toxicol., 18(7):460-70, 1999.

All of the compositions and methods disclosed and claimed herein can be made and executed without undue experimentation in light of the present disclosure. While the compositions and methods of this invention have been described in terms of exemplary embodiments, it will be apparent to those of skill in the art that variations may be applied to the composition, methods and in the steps or in the sequence of steps of the method described herein without departing from the concept, spirit and scope of the invention. More specifically, it will be apparent that certain agents that are both chemically- and physiologically-related may be substituted for the agents described herein while the same or similar results would be achieved. All such similar substitutes and modifications apparent to those of ordinary skill in the art are deemed to be within the spirit, scope and concept of the invention as defined by the appended claims. Accordingly, the exclusive rights sought to be patented are as described in the claims below.

1. A bifunctional pharmaceutical composition comprising at least one stress-responsive moiety operably linked to at least one active component having a diagnostic or therapeutic effect in an animal, wherein the at least one active component comprises at least a first diagnostic or therapeutic molecule.

2. The pharmaceutical composition of claim 1, wherein the at least one stress-responsive moiety is covalently linked to the at least one active component.

3. The pharmaceutical composition of claim 1, wherein the at least one stress-responsive moiety is operably linked to the at least a first diagnostic or therapeutic molecule.

4. The pharmaceutical composition of claim 1, wherein the at least one stress-responsive moiety is operably linked to a second diagnostic molecule, a second therapeutic molecule, or a combination thereof.

5. The pharmaceutical composition of claim 1, wherein the at least one stress-responsive moiety binds to a mammalian heat-shock protein.

6. The pharmaceutical composition of claim 1, wherein the at least one active component comprises a therapeutic molecule having a prophylactic effect.

7. The pharmaceutical composition of claim 1, wherein the at least one stress-responsive moiety binds to a protein or peptide that is induced, expressed, or upregulated in response to acoustic energy, radio frequency emission, or laser emission, or a combination thereof.

8. The pharmaceutical composition of claim 1, wherein the at least one stress-responsive moiety is responsive to heat-shock stress.

9. (canceled)

10. The pharmaceutical composition of claim 1, wherein the at least one stress-responsive moiety is operably linked to the at least one active component with a linker selected from the group consisting of:

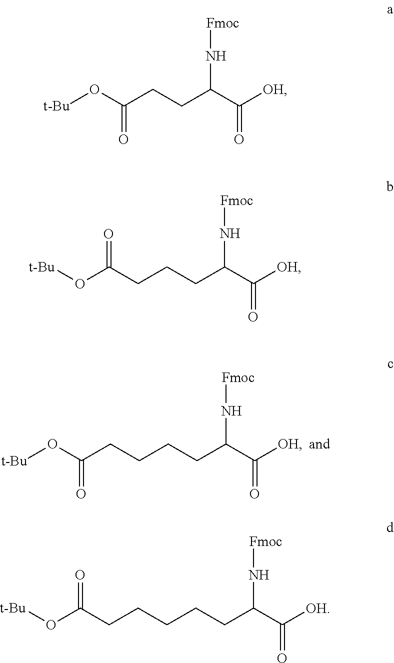

11. The pharmaceutical composition of claim 1, wherein the at least a first therapeutic molecule comprises doxorubicin, a compound of the formula: 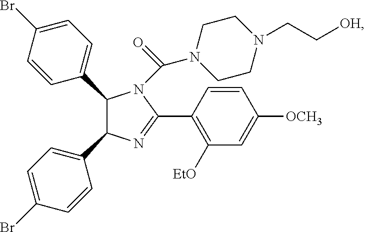
or an analog, derivative, or any combination thereof.

12. The pharmaceutical composition of claim 1, wherein the at least one stress-responsive moiety comprises a benzoquinone ansamycin, a near-infrared cyanine dye, a compound of the formula: 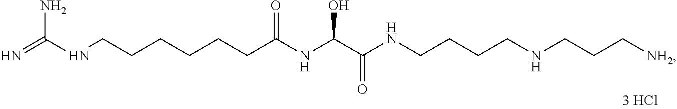
or an analog, derivative, or any combination thereof.

13. The pharmaceutical composition of claim 1, wherein the at least one stress-responsive moiety comprises Geldanamycin, a near-infrared Cyanine5.5 dye (Cy5.5), or an analog, derivative, or any combination thereof.

14. The pharmaceutical composition of claim 1, adapted and configured to release a portion of the at least one active component therefrom by application of heat, ultrasound, laser energy, photoacoustic energy, ultrasonography, light energy, radio frequency emission, a magnetic field, or a combination thereof.

15. The pharmaceutical composition of claim 1, wherein the at least one active component comprises one or more of an antineoplastic agent, an immunomodulating agent, a neuroactive agent, an anti-inflammatory agent, an anti-angiogenic agent, a chemotherapeutic, a radiotherapeutic, an antilipidemic agent, a receptor agonist or antagonist, and an antiinfective agent, or any combination thereof

16. The pharmaceutical composition of claim 1, wherein the at least one active component comprises one or more of a hormone, a protein, a peptide, an antibody, an antigen binding fragment, an enzyme, an RNA, a DNA, an siRNA, an mRNA, a ribozyme, a cofactor, and a steroid, or any combination thereof

17. The pharmaceutical composition of claim 1, wherein the diagnostic molecule comprises one or more of a detection agent, an imaging agent, a contrast agent, and a gas, or any combination thereof.

18. The pharmaceutical composition of claim 1, further comprising a liposome, a microbubble, a surfactant, a lipid complex formed from at least two different lipids, a niosome, an ethosome, a transferosome, a phospholipid, a sphingosome, or any combination thereof.

19. The pharmaceutical composition of claim 1, comprised within a nanoparticle, a microparticle, a nanocapsule, a microcapsule, a nanosphere, a microsphere, or any combination thereof.

20. The pharmaceutical composition of claim 1, formulated for administration to an animal host cell.

21. The pharmaceutical composition of claim 1, formulated for administration to a human host cell.

22-30. (canceled)

31. A method for localizing a therapeutic, diagnostic, or prophylactic compound to at least a first cell or a first population of cells within or about the body of an animal which comprises providing to an animal in need thereof a therapeutically-, diagnostically-, or prophylactically-effective amount of a bifunctional pharmaceutical composition that comprises at least one stress-responsive moiety operably linked to at least one active component having a diagnostic or therapeutic effect in an animal, wherein the at least one active component comprises at least a first diagnostic or therapeutic molecule, in the presence of a stress-inducing agent for a time sufficient to localize the composition to the at least a first cell or the first population of cells within or about the body of the animal.

32. A method for providing a diagnostic or imaging component to a selected population of cells or a first tissue site within or about the body of an animal which comprises providing to the animal an effective amount of a bifunctional pharmaceutical composition that comprises at least one stress-responsive moiety operably linked to at least one active component having a diagnostic or therapeutic effect in an animal, wherein the at least one active component comprises at least a first diagnostic or therapeutic molecule, in the presence of a stress-inducing agent under conditions effective to release the diagnostic or imaging component substantially only in the selected population of cells or first tissue site.

33. The method of claim 31, wherein the composition is administered to the animal either systemically or locally.

34. The method of claim 31, wherein the stress-inducing agent is locally provided to at least a first region of the body that includes the selected population of cells or first tissue site.

35. The method of claim 31, wherein the stress-inducing agent is locally provided to the at least a first region of the body by the application of laser energy, photothermal energy, photoacoustic energy, ultrasonography, magnetic resonance energy, radio frequency emission, infrared light, ultraviolet light, visible light, or heat.

36. The method of claim 31, wherein the animal is a mammal.

37. The method of claim 32, wherein the mammal is human.

38. The method of claim 32, wherein the composition is administered to the animal either systemically or locally.

39. The method of claim 32, wherein the stress-inducing agent is locally provided to at least a first region of the body that includes the selected population of cells or first tissue site.

40. The method of claim 32, wherein the stress-inducing agent is locally provided to the at least a first region of the body by the application of laser energy, photothermal energy, photoacoustic energy, ultrasonography, magnetic resonance energy, radio frequency emission, infrared light, ultraviolet light, visible light, or heat.

41. A method for providing a diagnostic or imaging component to a selected population of cells or a first tissue site within or about the body of an animal, comprising providing to the animal in the presence of a stress-inducing agent under conditions effective to release the diagnostic or imaging component substantially only in the selected population of cells or first tissue site, an effective amount of a bifunctional pharmaceutical composition that comprises at least one stress-responsive moiety operably linked to at least one active component having a diagnostic or therapeutic effect in an animal, wherein the at least one active component comprises at least a first diagnostic molecule, and further wherein the at least one stress-responsive moiety binds to a mammalian heat-shock protein or peptide.

42. The method of claim 41, wherein the composition is administered to the animal systemically, and the stress-inducing agent is locally provided to at least a first region of the body that includes the selected population of cells or first tissue site.

43. The method of claim 42, wherein the stress-inducing agent is locally provided to the at least a first region of the body by the application of laser energy, photothermal energy, photoacoustic energy, ultrasonography, magnetic resonance energy, radio frequency emission, infrared light, ultraviolet light, visible light, or heat.
